(19) United States
(12) Patent Application Publication
(10) Pub. No.: US 2024/0212846 A1
(43) Pub. Date: Jun. 27, 2024

(54) Protocol for a Cloud Based AI Clinical Decision Support System for Primary Care

(71) Applicant: Hussain Yusuf, Atlanta, GA (US)

(72) Inventor: Hussain Yusuf, Atlanta, GA (US)

(21) Appl. No.: 18/384,608

(22) Filed: Oct. 27, 2023

Related U.S. Application Data
(60) Provisional application No. 63/434,150, filed on Dec. 21, 2022.

(51) Int. Cl. G16H 50/20; G16H 50/70

(52) U.S. Cl. CPC G16H 50/20; G16H 50/70

(57) ABSTRACT

A system for recommending potential diagnoses, comprising memory; and one or more processors, wherein the one or more processors are configured at least to; receive user input indicating a plurality of clinical findings; determine that one or more first clinical findings of the plurality of clinical findings are normal clinical findings; determine that one or more second clinical findings of the plurality of clinical findings are abnormal clinical findings; determine a plurality of diagnostic scores for respective potential diagnoses of a plurality of potential diagnoses; and rank at least a portion of the plurality of potential diagnoses based at least on the plurality of diagnostic scores.

### TECHNICAL FIELD

The embodiments herein generally relate to systems, methods, and media for recommending potential diagnoses. More specifically, some embodiments herein are directed to cloud-based applications for clinical decision support for primary care and other related information needs by users (e.g., clinicians).

### BACKGROUND

The medical industry is complex. As complexity grows in, for example, the medical system in the United States, research data, disease data, new and advanced methods of disease treatment, and medical history data, the need for new and efficient systems to manage and apply data also grows.

Each year approximately 860,000,000 ambulatory care physician visits occur in the U.S.

Advances in medical knowledge and practice continually improve patient care by frontline clinicians, including internists, family practitioners, general practitioners, and nurse practitioners in outpatient and ambulatory care settings. However, the increasing body of medical care-related knowledge and evidence can pose a challenge to clinicians trying to consider many aspects during patient assessment and management.

Recent advances in informatics provide substantial opportunities to leverage extensive healthcare data (including EMR-based data) to create advanced analytic applications (including cloud-based platforms) and AI methodologies. This can provide systems and services to clinicians that can support their patient assessment and management activities.

Thus, there is an essential need for and utility of such applications. Current medical systems and resources often do not allow for fully leveraging the many data sources available today. Further, current medical systems and resources fail to efficiently connect and/or enable the optimal use of information by medical personnel involved in managing patients.

### SUMMARY

This summary intends to introduce various concepts in a simplified form that are further described in the detailed description of the embodiments. This summary does not intend to identify key or essential inventive concepts of the claimed subject matter. Therefore, this summary is not intended to be used for determining the scope of the claimed subject matter.

The embodiments herein generally relate to medical applications, systems, and methods. Some embodiments herein are directed to cloud-based applications for clinical decision support for primary care and other related information needs by users (e.g., clinicians). Some embodiments permit users to easily input patient information comprising patient-reported symptoms, clinical examination findings, lab results, imaging findings, chronic conditions, behavioral risk factors, family medical conditions, and/or patient demographics. In some embodiments, the inputted information is used for generating one or more digital reports that can support the clinician in his/her patient management actions.

In some examples, the embodiments herein permit users to easily input patient information (including patient-reported symptoms, clinical examination findings, lab results, imaging findings, chronic conditions history, behavioral risk factors, family medical history, and patient demographics) either by entering the information in multi-tab templates with drop-down lists of these information elements (with suggestions appearing based on partial text typed in by the user) and associated codes (e.g., International Classification of Diseases, 10th Revision, Clinical Modification [ICD-10-CM], LOINC, SNOMED, CPT, HCPCS) or by uploading (or dragging and dropping) a PDF or XML file that contains information about the patient. In some embodiments, users may review and approve the inputs by pressing a “Submit” button before the inputted data is used for generating a digital report.

In some embodiments, based on inputs about a patient, a digital report may be generated that includes a list of up to 25 diagnoses that scored highest per a scoring system accounting for one or more of the following factors:

1. The measurements of associations between all abnormal findings and potential diagnoses;

2. The measurements of associations between all normal findings and the ruling out of potential diagnoses;

3. The measurements of specificity of the associations between the abnormal findings and the diagnoses; and

4. The relative commonality of the potential diagnoses.

In some examples, a list of diagnoses may be ordered by their score values.

In some examples, for each diagnosis, the following information may be provided in a digital report: a clinical guidance document providing patient management information, lists of the most common (such as seen in a large sample of ambulatory outpatient visits) lab tests ordered, lab test results, imaging ordered, imaging findings, diagnostic procedures, diagnostic procedure findings, medications prescribed, therapeutic procedures, comorbidities, and patient care services associated with the diagnosis, as well as the titles, citations, and PubMed-abstract-weblinks to, for example, 10 journal articles with the highest metrics (e.g., Altmetrics) associated with the diagnosis.

In some embodiments, applications herein may enable users to select one or more diagnoses from drop-down lists of such diagnoses (with suggestions appearing based on partial text typed in by a user), which may be used to generate digital reports for each of the diagnoses. Each digital report may contain the same type of information for the respective diagnosis as the digital reports described herein.

Some embodiments described herein may be compatible with large healthcare data sources comprised of electronic medical records, healthcare claims, and/or health system administrative data sources. Such data sources can include real patient data and/or synthetic data.

The data acquired and used in the cloud-based applications herein may be stored in a data repository. The repository may be updated with more recent data as such are received.

Cloud-based applications described herein may be helpful to the clinician by enabling her or him to identify a set of diagnoses that are potentially associated with a patient's clinical findings and available clinical support information for those diagnoses, including common lab tests, imaging, procedures, and medications and allow recommendation of patient care services associated with the respective diagnoses. Embodiments herein also make available clinical guidance material and link to high-metric journal publications related to diagnoses.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 illustrates a block diagram of a system for recommending potential diagnoses, according to one or more aspects.

FIG. 2 illustrates a flow diagram of a process for recommending potential diagnoses, according to one or more aspects.

FIG. 3 illustrates a flow diagram of a process for determining a diagnostic score for a potential diagnosis, according to one or more aspects.

FIG. 4 is a schematic of an application, according to one or more aspects.

FIGS. 5-20 are example graphical user interfaces associated with an application, according to one or more aspects.

### DETAILED DESCRIPTION

Any specific details of features or aspects are used for demonstration purposes only, and no unnecessary limitations or inferences are to be understood therefrom.

Before describing in detail exemplary aspects, it is noted that the aspects reside primarily in combinations of components and procedures related to the system and/or apparatus. Accordingly, the system and/or apparatus components have been represented where appropriate by conventional symbols in the drawings, showing only those specific details that are pertinent to understanding the aspects of the present disclosure so as not to obscure the disclosure with details that will be readily apparent to those of ordinary skill in the art having the benefit of the description herein.

As used herein, relational terms, such as “first” and “second,” “top” and “bottom,” and the like, may be used solely to distinguish one entity or element from another entity or element without necessarily requiring or implying any physical or logical relationship, or order between such entities or elements. Furthermore, there is no intention to be bound by any expressed or implied theory presented in the preceding technical field, background, summary, or the following detailed description. It is also to be understood that the specific devices and processes illustrated in the attached drawings, and described in the following specification, are simply exemplary aspects of the inventive concepts defined in the appended claims. Hence, specific steps, process order, dimensions, component connections, and other physical characteristics relating to the aspects disclosed herein are not to be considered as limiting, unless the claims expressly state otherwise. The use or mention of any single element contemplates a plurality of such element, and the use or mention of a plurality of any element contemplates a single element (for example, “a device” and “devices” and “a plurality of devices” and “one or more devices” and “at least one device” contemplate each other), regardless of whether particular variations are identified and/or described, unless impractical, impossible, or explicitly limited.

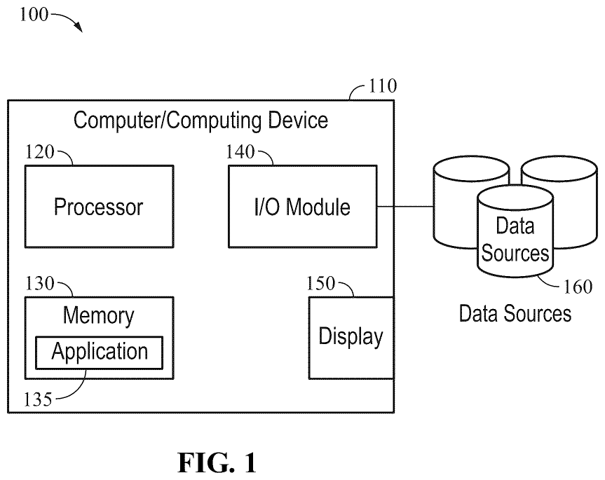

FIG. 1 illustrates a system 100 for determining one or more potential diagnoses, according to one or more aspects. Various features described within FIG. 1 may generally complement the description of the other figures of the present disclosure. The system 100 for determining one or more potential diagnoses includes a computer 110, which includes one or more processors 120, a memory 130 including an application 135, an input/output module (or “I/O module”) 140 connected directly or through a network (not shown) to data sources 160, and a display 150.

The one or more processors 120 is wired and/or wirelessly connected to and/or communicatively connected to the memory 130 and the I/O module 140. The I/O module 140 is wired and/or wirelessly connected to and/or communicatively connected to the display 150 and the network and/or to the data sources 160. A computing device such as the computer 110 can be or be embedded in another device, e.g., a mobile telephone, a smartphone, a tablet, a laptop, server, desktop computer, a personal digital assistant (PDA), a portable storage device (e.g., a universal serial bus (USB) flash drive), and/or the like. Likewise, a general computer or computing device may become a system 100 for determining one or more potential diagnoses via the execution of an application such as the application 135.

Referring to FIG. 1, the one or more processors 120 includes any suitable processor implemented as a central processing unit (CPU), a graphics processing unit (GPU), an application-specific integrated circuit (ASIC), a field programmable gate array (FPGA), an artificial intelligence (AI) accelerator, any other type of processing unit, or a combination of different processing units, such as a CPU configured to operate in conjunction with a GPU. In general, the one or more processors 120 may be any technically feasible hardware unit capable of processing data and/or executing software applications such as the application 135. Further, in the context of this disclosure, the computing elements shown in the computer 110 may correspond to a physical computing system (e.g., a local or networked computing device) or may be a virtual computing instance executing within a computing cloud.

Memory 130 includes a random-access memory (RAM) module, a read-only memory (ROM) module, a flash memory unit, a hard disk, magnetic disks, optical disks, magneto-optical disks, a mass storage device, a database, and/or any other type of memory unit or combination thereof. The one or more processors 120 and I/O module 140 are configured to read instructions and/or data from and write instructions and/or data to memory 130 and/or the data sources 160. Memory 130 stores various software programs that can be executed by processor(s) 120 and application data associated with said software programs, including software that runs methods, steps, and processes such as the software, methods, steps, and/or processes described in this disclosure. The various software programs and the associated application data include the application 135.

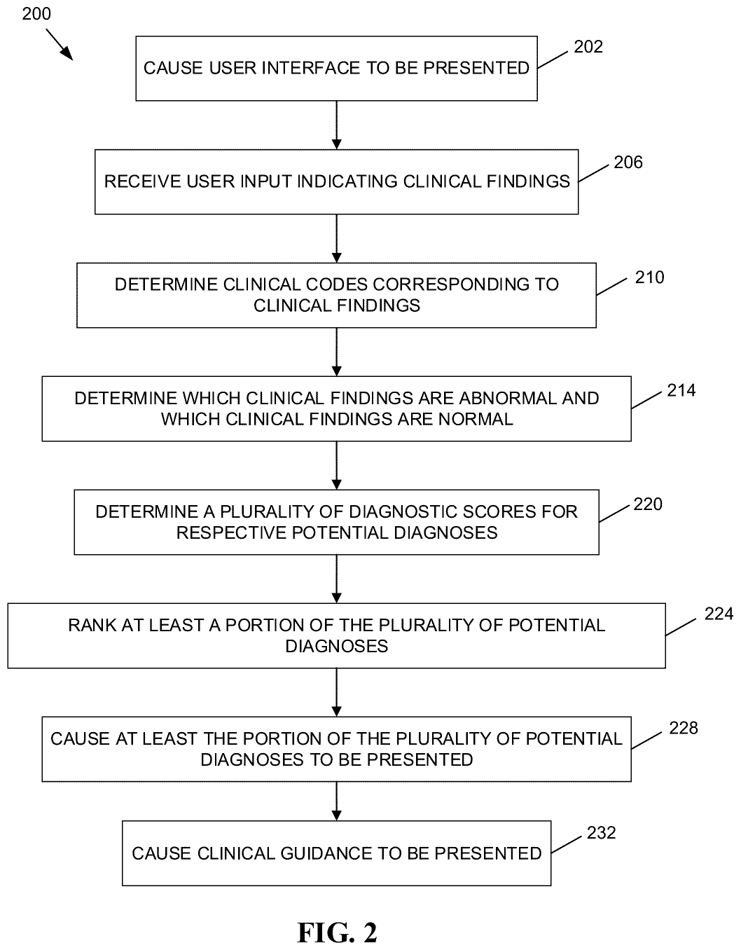

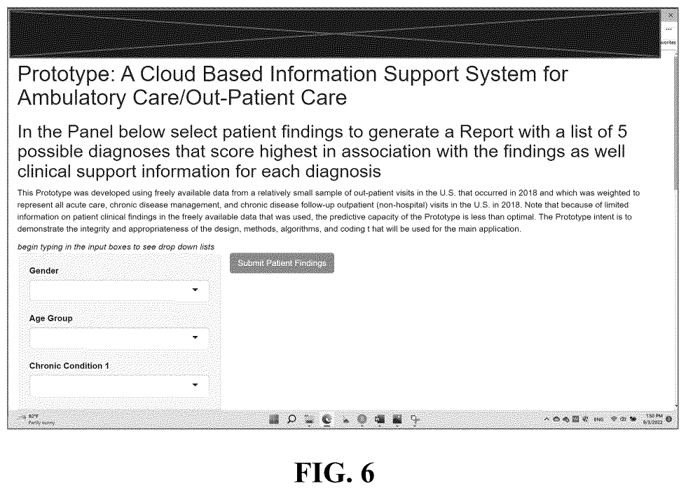

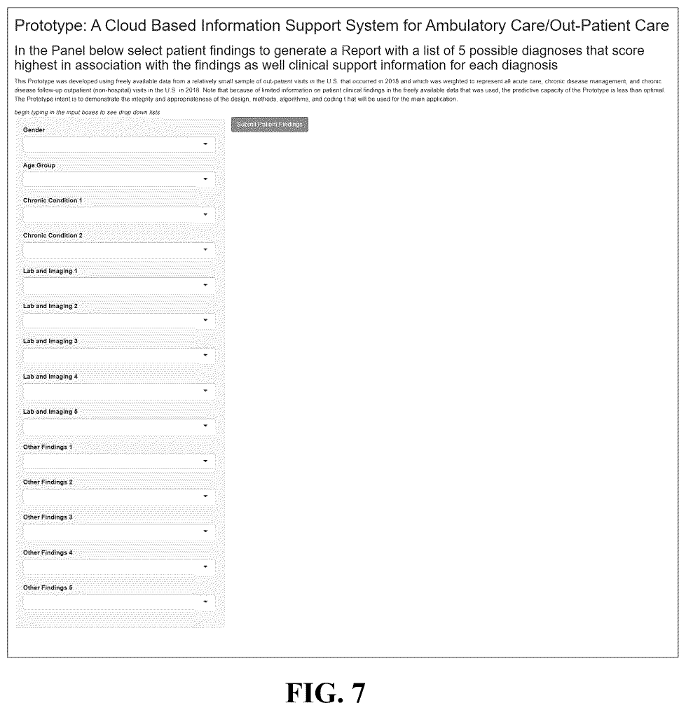

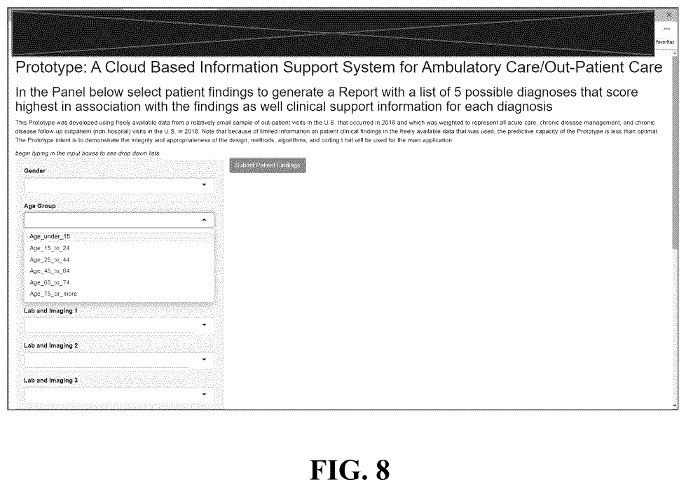

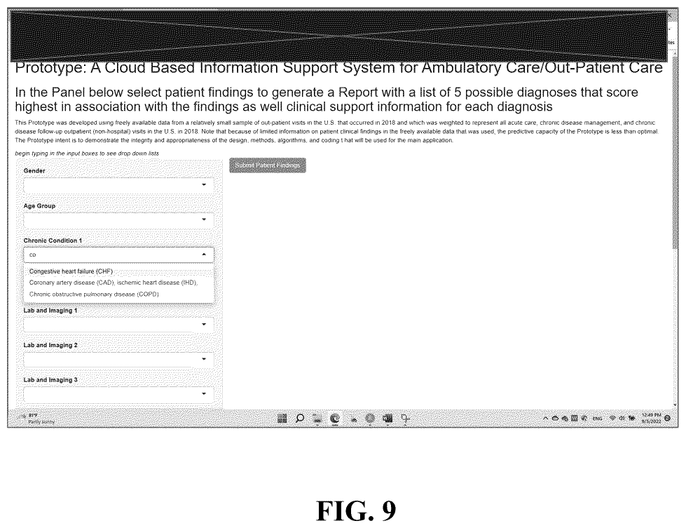

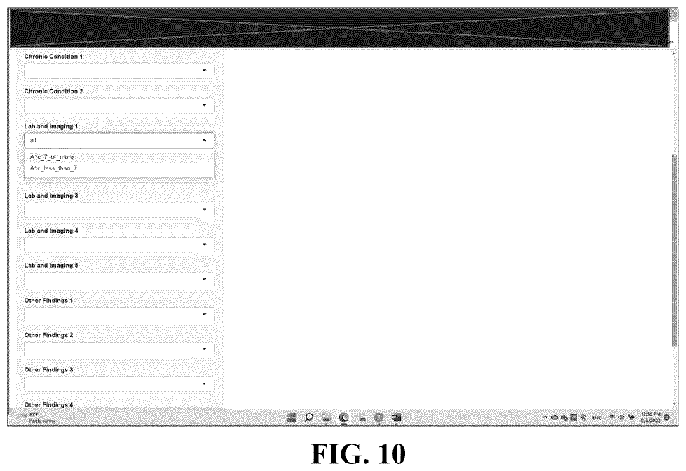

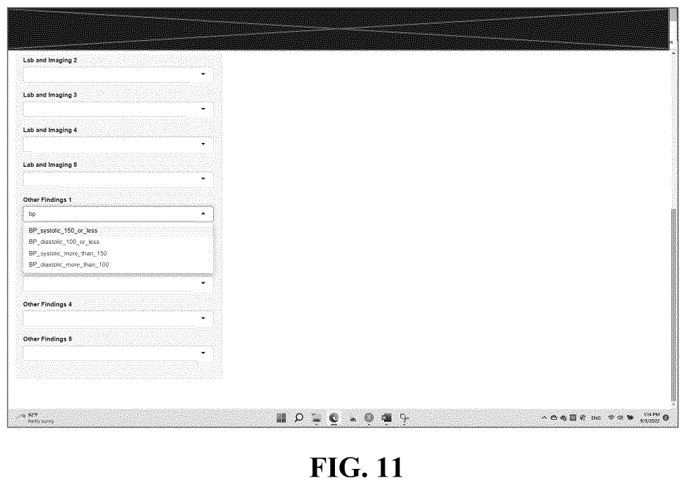

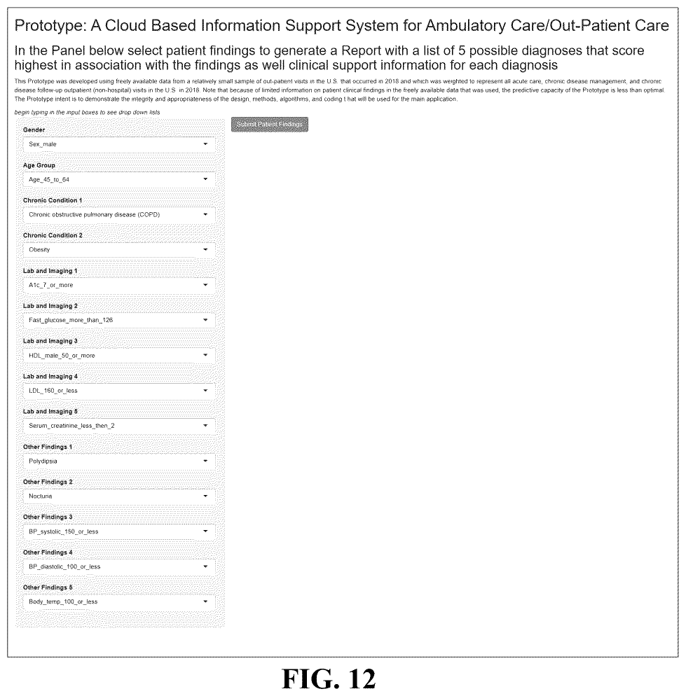

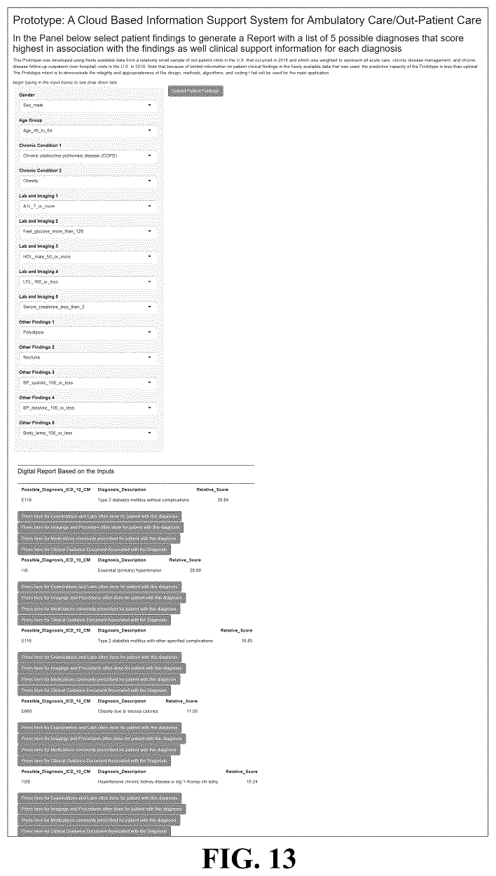

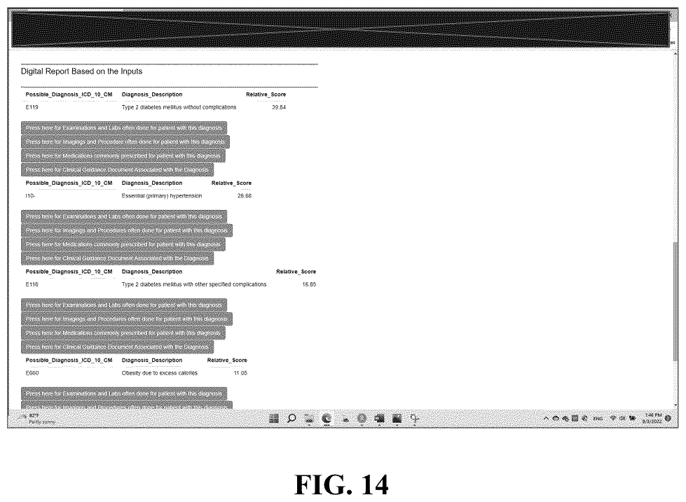

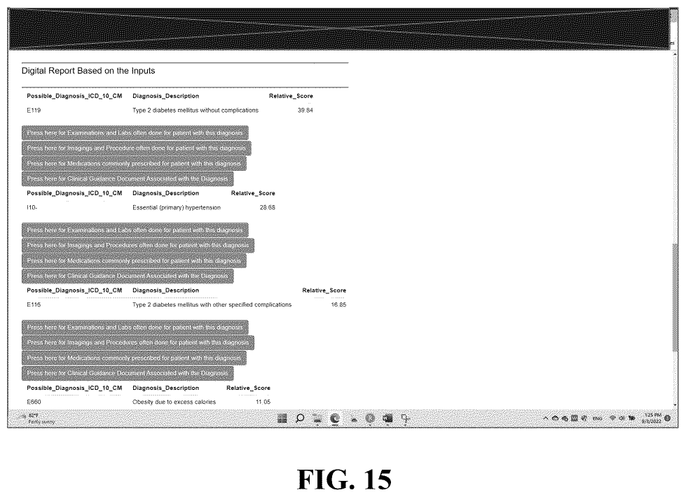

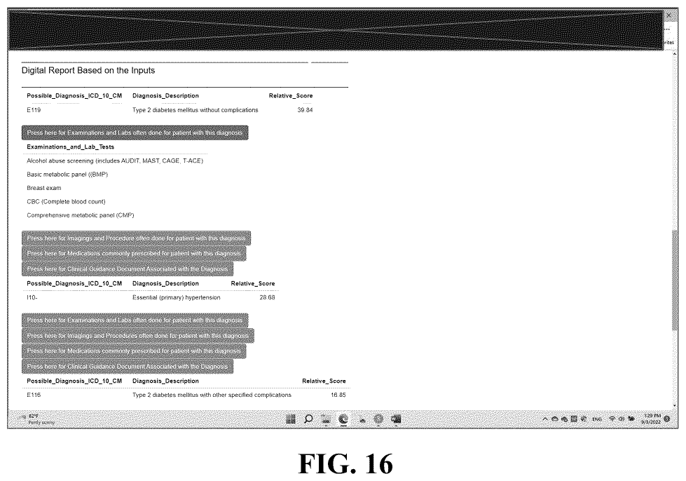

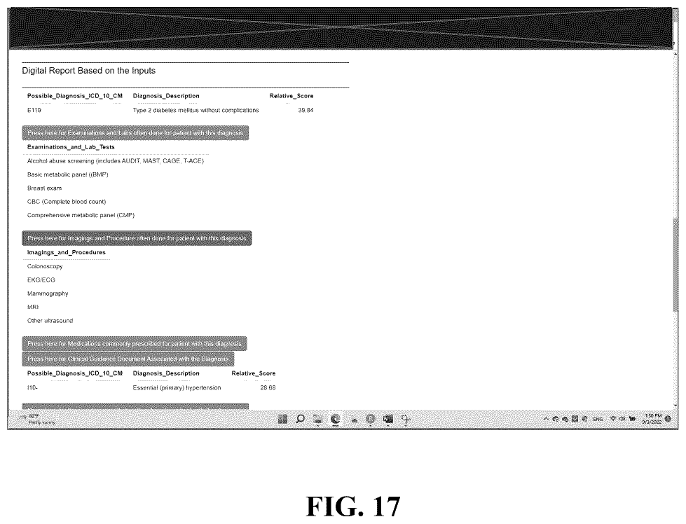

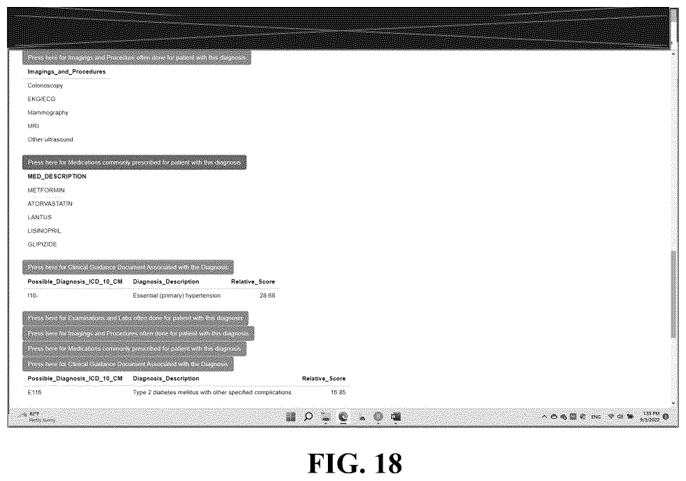

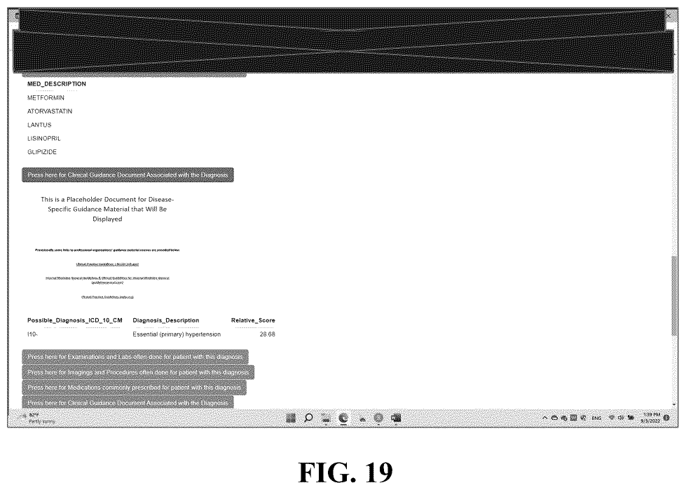

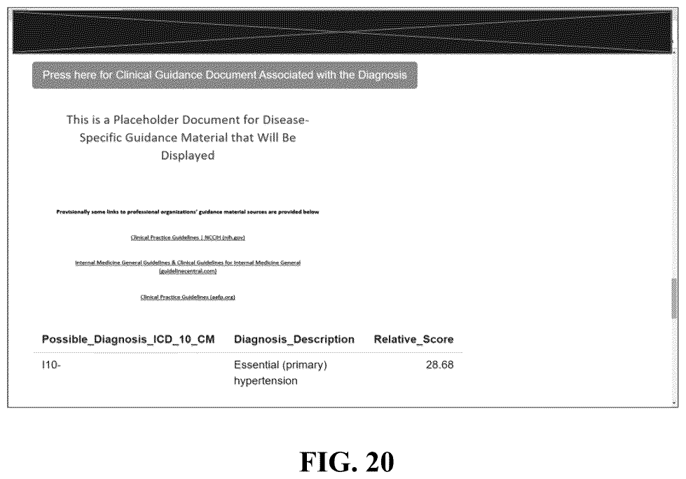

The application 135 is configured to implement aspects described herein. The memory 130 and the data sources 160 store data accessible by the application 135. According to one or more aspects, the application 135 may include software elements corresponding to one or more of the various aspects described herein. For example, application 135 may be implemented in various aspects using any desired programming language, scripting language, or combination of programming languages and/or scripting languages (e.g., C, C++, C#, PYTHON®, JAVA®, JAVASCRIPT®, PERL®, R, and/or the like). Furthermore, the code, instructions, data, functionality, and/or results from processing and/or execution of the application 135 can vary according to the person or use of the system, the data available, the device and/or user running the application (for example, the application 135 in a server, an administrator computer, a staff/employee computer, and a client or user computer), the role of the user and/or the device (for example, the user of a computer running the application 135 as an administrator then switching and using the computer running the application 135 as a client), and/or the like. Therefore, the application 135 may have aspects that operate in different computers that may or may not be connected to each other and/or to the same network(s). Aspects of the application 135 are further described in the description of FIGS. 2-20, below.

The I/O module 140 provides the hardware, firmware, and/or software for the computer 110 to interact with other components and devices, including the data sources 160, the display 150, the network, other devices connected to the network, and/or the like. According to one or more aspects, the I/O module 140 can be connected to other devices, either wired or wirelessly, including input peripherals such as keyboards, microphones, cameras, and/or the like. According to one or more aspects, the I/O module 140, automatically and/or manually via user input through the display 150 and/or via the network, request, obtain, get, post, put, create, read, update, and/or delete any information and/or data from other computers and/or devices connected to the network. The one or more processors 120, through the I/O module 140, actuates, creates, generates, sends, and/or receives signals and/or data to and/or from the network and/or the display 150. The I/O module 140 is configured to allow data to be exchanged between the computer 110 and other devices attached to a network, such as other computer systems, cloud services, remote databases, and/or the like. According to one or more aspects, the I/O module 140 supports communication via wired or wireless general data networks, such as any suitable type of Ethernet network, for example, via telecommunications/telephony networks such as analog voice networks or digital fiber communications networks, via storage area networks such as Fiber Channel SANs, or via any other suitable type of network and/or protocol, including WAN, LAN, Wi-Fi®, Bluetooth®, and/or the like.

Cloud services are any networked, online, internet, and/or web services that may be implemented or used to perform as or embody part or all of the computer 110 and/or to execute part or all of the application 135, handle and/or store the data accessed by the application 135, via provisioning software, Software-as-a-Service (SaaS), platform, Platform-as-a-Service (PaaS), infrastructure, Infrastructure-as-a-Service (IaaS), artificial intelligence models and/or processing, Artificial Intelligence-as-a-Service (AlaaS), and/or other technologies, such as Amazon Web Services (AWS®), Microsoft® Azure®, Google Cloud Platform (GCP®), Snowflake®, Heroku®, Netlify®, DigitalOcean®, IBM® Cloud, Oracle® Cloud Infrastructure, and/or the like.

The display 150 includes devices capable of providing input, such as a touchscreen, touch sensitive display, keyboard, a mouse, a smart pen, a camera, buttons, a microphone, a headphone, a headset, photosensors, biosensors, haptic sensors, and so forth, as well as devices capable of providing output, such as a display device, a touchscreen, a touch sensitive display, a speaker, headphone, a headset, touchscreen, haptic transducer, LED's, and so forth. Additionally, the display 150 may include devices capable of both receiving input and providing output, such as a touchscreen, a touch sensitive display, haptic devices, a universal serial bus (USB) port, and so forth. The display 150 may be configured to receive various types of input from a user, such as verbal commands, typed input, gestures, sounds, and so forth, and to also provide various types of output to the user, such as displayed digital images, digital videos, sound, text, alarms, reminders, haptic sensations, and so forth. According to one or more aspects, the display 150 (or part of the display 150) is embedded and/or integrated with the computer 110. For example, the computer 110 may have the display 150 on the housing that encloses the computer 110. According to one or more aspects, the display 150 (or part of the display 150) is separate from (is not embedded and/or integrated with) the computer 110. For example, the computer 110 may have the display 150 separate from the housing that encloses the computer 110, while connected wirelessly to the computer 110 via Bluetooth® or another wireless protocol.

The computer 110 and/or the data sources 160 may be local, remote, cloud-based, implemented via cloud services, shared, dedicated, and/or the like. The data sources 160 includes a computer, a database, a data warehouse, a data lake, a data repository, a file storage, RAM, ROM, flash memory, and/or the like. The data sources 160 can be or include any database technologies and/or any database management system (DBMS), including hierarchical, network, relational, object-oriented, sql, no-sql, graph, ER model, and/or document databases and/or DBMSs, and/or the like. Throughout the description of the various aspects, it should be understood that the memory 130 and/or the data sources 160 store and allow reading, writing, editing, and/or deleting of any data, values, information, parameters, and/or the like, for the operation of the computer 110, application 135, and/or the system 100 for determining one or more potential diagnoses. According to one or more aspects, the data sources 160 is a plurality of databases. According to one or more aspects, the data sources 160 is implemented or resides partially or completely inside of the computer 110 and/or are accessible to the computer 110 and/or the application 135 via the network.

The network includes any technically feasible type of communications network that allows data to be exchanged between the computer 110 and external entities or devices. For example, network may include a wide area network (WAN), a local area network (LAN), a wireless (Wi-Fi®) network, Bluetooth®, and/or the Internet, among others (including any communication channel, medium, and/or network accessible by the I/O module 140). According to one or more aspects, the I/O module 140 is not directly connected to one or more elements, parts, components, devices and/or networks. According to one or more aspects, the I/O module 140 connects wired and/or wirelessly to the to one or more elements, parts, components, devices and/or networks through the network.

Also, any connection may be associated with a computer-readable medium or media. For example, the application 135 and/or data associated with or operated by the application 135 may be transmitted from or through a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave, all of which are included in the definition of medium. Storage of any kind data of the application 135 and/or data associated with or operated by the application 135 such as hard discs, disks, RAM, ROM, flash memory, cloud storage, and/or the like (including the memory 130 and/or the data sources 160) is also included in the definition of medium. “Disk” and “disc,” as used herein, include compact disc (CD), laser disc, optical disc, digital versatile disc (DVD), floppy disk and Blu-ray disc where disks usually reproduce data magnetically, while discs usually reproduce data optically with lasers. Combinations of the above should also be included within the scope of computer-readable media. The computer-readable medium can be transitory or non-transitory.

The data sources 160 include one or more databases or electronic sources of patient data or any other data used or received by the application 135. Patient data is any data, whether historic data, real-time data, delayed data, stored data, and/or the like, about one or more patients (whether real or hypothetical), any data that can be used as patient data, or any data that can be used to diagnose any patient, including for example, any clinical findings, or any data in any table disclosed herein (e.g., Tables AAA0 to AAA7, BBB0 to BBB3, CCC1 and CCC2, and FFF1 TO FFF5).

### Development Phase/Review and Upkeep Phase

In some embodiments, resource-intensive processes (e.g. computer processing unit [CPU] usage) may be frontloaded to the development phase of the application 135 to provide an efficient and highly responsive system 100 when the developed application is used by its intended users. For example, in some embodiments, the application 135 can be designed so that many processes and subprocesses associated with related patient's clinical findings, probable diagnoses, and common patient management actions is not repeated and/or carried out at the time the application 135 is used (such as when users access and use the application 135). In some embodiments, the application 135 can be a cloud-based application 135.

In some embodiments, processes may be performed using statistical computing and programming languages such as R, SAS, SQL, and/or Python. In some embodiments, the processes can be performed using cloud-based data platforms and applications.

Various steps may be performed during the development of data and data tables that may be drawn upon when users (e.g., clinical providers) use the application 135 (e.g., when users input patient information to generate reports). In some embodiments, these steps need not be repeated when users (e.g., clinicians) interact with the application 135 and during the generation of reports. Instead, these steps may be completed prior to the launch of the application 135 and thereafter may be reviewed and updated (as needed) periodically (e.g., quarterly).

In some embodiments, various data and data tables created by such steps may be stored in data repositories and used for application purposes including (but not limited to):

1. Acquiring, storing, and mapping user inputs through a user interface related to patient characteristics;

2. Developing metrics conveying measurements of a positive and negative association between various clinical findings and diagnoses, the specificity of associations, and the commonality of diagnoses;

3. Generating possible diagnoses and associating clinical support information based on user inputs about the patient;

### Development of Data Tables Storing Disease, Clinical Findings, Lab Tests and Results, Imaging and Results, Medication, and Other Disease Explanatory Factor Codes and Descriptions

Various data tables may be created and/or developed according to embodiments herein to operationalize the use of the application 135 by users to generate digital reports (with potential diagnoses and clinical support information). There will be periodic assessment and updating of data, algorithms, and processes associated with the application 135.

Tables may hold data for associating patient-related user inputs (e.g., indicating clinical findings) with potential diagnoses (e.g., ICD-10-CM-based diagnoses of diseases and disease conditions). Such table development may be via a multi-step process. These tables may be stored as a partitioned data source to improve computing efficiency.

In some examples, a set of tables may be created, identified herein for example purposes as Tables AAA0, AAA1, AAA2, AAA3, AAA4, AAA5, AAA6, and AAA7.

### Example Table AAA1 and Interim Data Pools

A table referred to as “Table AAA1,” for example, may be developed that contains patient characteristics, clinical findings, and patient visit information from a large sample of healthcare visits. Observations in Table AAA1 may be at the outpatient ambulatory care visit level (i.e., one observation and/or line per one outpatient ambulatory care visit may be included).

In another example, data from the most recent years (e.g., most recent 5 years) from large healthcare data sources (e.g., electronic medical records data, healthcare claims-based data, and/or healthcare administrative data) that includes (or can be used to develop) line-level patient-health visit information may be used for developing Table AAA1. The criteria for selecting and acquiring large data sets may include the data having patient details regarding one or more of the following: patient demographics, patient health problems, clinical findings, lab tests ordered lab results, imaging and other diagnostic procedures conducted and their results, surgical and therapeutic procedures conducted, chronic conditions, behavioral risk factors, family medical history, primary and secondary diagnoses, medications prescribed and/or administered, as well as other patient management information.

Potential data sources for creating Table AAA1 include (but may not be limited to) commercially available electronic medical records, claims, and/or administrative data sources, which may include real patient data as well as synthetic data. The data acquired for use with the application 135 herein may be stored in a data repository. The repository may be updated with more recent data as such are obtained.

### Interim Data Pools

Data from one or more data sources described above (e.g., electronic medical records data acquired from data vendors) may be used to form an interim pool of data. From this interim pool, an interim table (e.g., “Table A-Interim”) of line-level data at the health care visit level that meets a required level of completeness (e.g., having SNOMED, LOINC, and ICD-10 code based information on diagnoses, clinical findings, lab test, lab test results, imaging, imaging findings, patient risk factors, diagnostic procedures, medications, therapeutic procedures, and patient care services) may be developed. Observations in Table A-Interim may include the following variables:

1. Patient age, gender, race, ethnicity, date of healthcare visit, provider type, type of healthcare visit, type of healthcare facility, locations of healthcare facility (e.g., region, division, state [if available]), discharge date, and discharge status. Additionally, individual variables may be present for each of these data elements;

2. The diagnosis, including the diagnosis code (e.g., ICD-10-CM code) for the primary diagnosis;

3. All diagnoses, including diagnosis codes (e.g., all ICD-10-CM codes) for the secondary diagnoses—these may be included multi-data-element variables where the codes may be separated by separators (e.g., commas or pipes);

4. Multi-data element fields for each of the following types of information: clinical findings, diagnostic procedures and results, laboratory tests and test results, imaging and imaging results, medications, therapeutic procedures, patient care services, chronic conditions, family medical history, and socio-behavioral risk factors;

During the creation of Table A-Interim, visit level observations from each contributing data source that includes the data elements described for Tables AAA1-AAA3 herein may be linked together using patient and visit related identifiers available in the source data.

A sample may then be drawn from Table AAA1-Interim to form Table AAA1. During the development of Table AAA1, it may be ensured that the data in Table AAA1 is representative of the outpatient ambulatory healthcare visits that occur nationally and, to the extent possible, at sub-national levels (e.g., census region or division) and/or the data in Table AAA1 may include observation level weights that can be applied during the analysis to produce nationally (and possibly, sub-nationally) representative results. Criteria for national representativeness of the data may be based on the distribution of selected patient and healthcare visit characteristics among a weighted national sample of outpatient ambulatory care visits captured in other available data on representative samples of ambulatory care visits. The criteria by which representativeness may be determined may include patient demographics (e.g., gender, age, race, ethnicity), patient location information (e.g., region), provider type, and diagnosis-related information (e.g., principal diagnosis, DRG, etc.).

Each observation in Table AAA1 may have a unique identifier that can be used to link it to the corresponding observation in the original data source and an identifier that can be linked to the observation in Table AAA0 that may house metadata about the data source from which the observation is derived. Sampling techniques (e.g., through procedures such as sample, samplePPS, match, and rbind procedures in R, select, join, like commands in sql, proc surveyselect in SAS,) may be applied to carry out the steps described above. The resultant data may be stored as a partitioned data set, Table AAA1, in a data repository and may be updated quarterly using the most recent available data.

### Example Table AAA2

A table referred to as “Table AAA2,” for example, may be created that may be a repository of some or all commonly used standard medical coding (e.g., ICD-10, SNOMED, LOINC, CPT, and HCPCS codes). Each unique code may be entered into a unique observation. Codes for which observations may be created in Table AAA2 may be clinical and other patient findings codes, e.g., SNOMED codes that convey the findings from an evaluation (including e.g., patient symptoms and signs), examination, lab test, etc. (observations may not be created for parent codes which do not convey findings). This may be done first for all codes where the code itself represents the clinical finding. For clinical findings codes that report the findings through a separate quantitative or qualitative data element (e.g., many LOINC codes do so), the observations in Table AAA2 may have some additional characteristics, as described herein).

Each observation in Table AAA2 may include (among others) the following data elements: the code set identifier, the code, short description of the code, long description of the code, parent codes (as a multi-data element field), child codes (as a multi-data element field), and the year and month the code became effective. In addition, each observation in Table AAA2 may have a 0/1 (no/yes) variable indicating whether the finding is an abnormal finding and a 0/1 variable for whether it is a normal finding. For example, all SNOMED finding codes that are indicated to be normal findings in the code description or have an associated qualifier code (e.g., indicating normal findings or the findings being within the normal range) may have a value of 1 for the normal finding variable and zero for the abnormal finding variable. Table AAA2 may also include observations for each age group (e.g., 10 year age groups), male and female sex (or gender), race group, ethnicity group, and patient location (e.g., census regions or divisions, states), with a 0/1 code being assigned to the presence or absence of each characteristic (e.g., male sex). In addition, each observation in Table AAA2 may include the following 0/1 (No/Yes) variable: whether the code represents a diagnosis of a disease or disease condition, a clinical evaluation finding, a lab test, a lab test result, an imaging, an imaging result, a diagnostic procedure (other than a lab test or imaging), a diagnostic procedure result, a surgical or therapeutic procedure or patient care related service, a medication, or none of these. In some embodiments, observations for diagnosis codes may be indicated as representing a diagnosis as well as a clinical finding. In some embodiments, codes associated with findings from lab tests, imaging, and other diagnostic procedures may be indicated as clinical findings. In some embodiments, codes for demographics such as, for example, age group, sex (or gender), race group, ethnicity group, and patient location may be indicated as clinical findings and as normal findings (this may be needed for their use in the predictive algorithms of this application). In some embodiments, a normal clinical finding can be a clinical finding that is expected or typical for a healthy patient. In some embodiments, a normal clinical finding can be a clinical finding that is within a predetermined range for a healthy patient. In some embodiments, codes representing behavioral and other risk factors may be indicated as being clinical findings and as being abnormal findings. In some embodiments, an abnormal clinical finding can be a clinical finding that is not expected or not typical for a healthy patient. In some embodiments, an abnormal clinical finding can be a clinical finding that is not within a predetermined range for a healthy patient. Each observation in Table AAA2 may have a unique identifier for the observation (referred to hereafter also as AAA1-Identifier). Table AAA2 may be stored in a data repository and updated annually as appropriate.

For codes (e.g., many LOINC codes do so) that report test results using a separate variable that provides a quantitative value, there may be three corresponding observations in Table AAA2. Each of these three observations may include all data elements already described above (for codes where the code itself is the clinical finding). In addition, each of these three observations may include a data element that differentiates it from the other two in the context of the clinical finding value, with one observation representing a higher-than-the-normal range finding, one observation representing a lower-than-the-normal range finding, and one observation representing a normal range finding. In some embodiments, during development and tuning of the application 135, further stratification may be considered for many of these codes. In addition, all three of these observations may include two other data elements, one data element indicating the lower bound of the normal range of the clinical finding and one data element indicating the upper bound of the normal range of the clinical finding.

For codes (e.g., many LOINC codes do so) that report test results using a separate variable that provides a qualitative value, there may be two corresponding observations in Table AAA2. Each of these two observations may include all data elements already described above (for codes where the code itself is the clinical finding). In addition, each of these two observations may include a data element that differentiates it from the other in the context of the clinical finding value, with one observation representing the positive clinical finding of the test (e.g., organism detected) and one observation representing the negative finding (e.g., not detected).

For observations in Table AAA2 for medications, the code representing the observation may be the unique AAA6-identifier code for the medication that is present in a medications cross-walk table (such as Table AAA6, as described herein). In addition to the other variables described for the observations in Table AAA2, each medication-related observation may also have variables for the following: NDC code, the NDC code name, the RxNorm code, the RxNorm name; the RxCIU code, the RxCIU code name, the GPI subclass code, and the GPI subclass code name. In observations in Table AAA2 that are not for medications, these variables may be set to null.

### Example Table AAA3

A table referred to as “Table AAA3,” for example, may be created where the line level (e.g., based on event—health care visit) data from Table AAA1 may be included as observations in Table AAA3. The ICD-10-CM coded primary diagnosis from each observation in Table AAA1 may be entered as the same into the corresponding observation in Table AAA3. All secondary diagnoses and clinical findings from each observation in Table AAA1 may be entered into multi-element data fields in the corresponding observation in Table AAA3.

When transferring information from an observation in Table AAA1 to the corresponding observation in Table AAA3, each clinical finding (e.g. patient problems, clinical findings, laboratory test results, imaging findings, family-behavioral-sociodemographic-medical history based risk factors, and chronic conditions), test, procedure, and service that are available as a coded data element (e.g. entered using ICD-10, SNOMED, LOINC, CPT, and HCPCS codes) in Table AAA1 may be mapped to Table AAA2 and the corresponding AAA1-identifier code may be entered into one of the following multi-data-element variables in Table AAA3: abnormal clinical finding/normal clinical finding/lab tests ordered/imaging and other diagnostic procedure ordered/medications/surgical procedures, therapeutic, and medical care related services. The multi-element data fields may be variables where AAA1-identifier codes may be entered (separated by separators such as pipes or semicolons). Information on sex (or gender), age group, race group, ethnicity group), and patient location in Table AAA1 may also be mapped to Table AAA2, and the corresponding AAA1-identifiers may be entered into Table AAA3 in the normal clinical findings field. Each observation in Table AAA3 may have a unique identifier assigned to it. Each observation in Table AAA3 may also have an identifier that links it to the corresponding observations in Table AAA1 and an identifier that links it to metadata information about the original data source that contributed to Table AAA0. Table AAA3 may be stored as a partitioned data set in a data repository and updated quarterly using the most recently available data.

When transferring observation level data from an observation in Table AAA1 to the corresponding observation in Table AAA3, each clinical finding that is present in Table AAA1 in the form of a code (e.g., a LOINC code) that reports the finding via a separate quantitative data element, the code, and the quantitative value may be mapped first from Table AAA1 to the appropriate one of the three corresponding observations that exist in Table AAA2 based on whether the reported value is below, within, or above the normal range values for that clinical finding.

When transferring observation level data from an observation in Table AAA1 to the corresponding observation in Table AAA3, each clinical finding present in Table AAA1 in the form of a code (e.g. a LOINC code) that reports the finding via a separate qualitative data element, the code, and the qualitative value may be mapped first from Table AAA1 to the appropriate one of the two corresponding observations that exist in Table AAA2 based on whether the reported value is a positive clinical finding (e.g., organism detected) or a negative clinical finding (e.g., not detected). R, SAS, Sql, and/or python programming language can be used to carry out the above steps.

### Example Table AAA4

A table, referred to as “Table AAA4,” for example, may be developed, which may be a cross-walk of all codes (e.g., ICD-10-CM, SNOMED, and LOINC codes) that represent the same clinical findings (including patient signs and symptoms, diseases, and test, imaging, and procedure based findings). The codes from all observations included in Table AAA2 that are indicated to be clinical findings codes may be included in Table AAA4. Each observation may also have a multi-data-element variable that may house all AAA1 identifiers that indicate the same clinical finding, with individual identifiers separated by a separator (e.g., a comma or pipe). Each observation in Table AAA4 may also include a unique identifier (hereafter referred to as AAA4-identifier).

### Example Table AAA5

A table, referred to as “Table AAA5,” for example, may be developed, which may be a cross-walk of all codes (e.g., CPT, HCPCS, SNOMED, and LOINC codes) that represent the same lab tests, imaging, other diagnostic procedures. The codes from all observations included in Table AAA2 that are indicated to be lab tests, imaging, and other diagnostic procedures (as per the value for that data element in Table AAA2) may be included in Table AAA5. Each observation may have a multi-data-element variable that may house all AAA1 identifiers that indicate the same lab tests, imaging, or other diagnostic procedures, with individual identifiers separated by a separator (e.g., a comma or pipe). Each observation in Table AAA5 may also include a unique identifier (hereafter referred to as AAA5-identifier).

### Example Table AAA6

A table, referred to as “Table AAA6,” for example, may be developed that may be a cross-walk for all medication codes represented in different classification systems, including (but not limited to) the National Drug Code (NDC), RxNorm code, HCPCS medication codes, and MULTUM drug codes. Each observation in Table AAA6 may represent a different NCD drug code. Each observation in Table AAA6 may have individual variables for the following data elements: the NDC code, the NDC code description, the corresponding RxNorm, HCPCS, and MULTUM codes and their descriptions, the corresponding RxNorm Concept ID (RxCIU) and its description, and the corresponding Generic Product Identifier (GPI) codes and associated descriptions for the drug name, sub-class, class, and group, and a link to web-based information about the drug. Each observation in Table AAA6 may have a unique identifier named AAA6-identifier.

### Example Table AAA7

A table, referred to as “Table AAA7,” for example, may be developed that may be a cross-walk for all codes (e.g., CPT, ICD-10, HCPCS) that represent surgical, therapeutic, and medical care-related services. The codes from all observations included in Table AAA2 that are indicated to be surgical, therapeutic, or medical care services-related codes (as per the value for that data element in Table AAA2) may be included in Table AAA7. Each observation may have a multi-data-element variable that may house all AAA1 identifiers that indicate the surgical, therapeutic, or medical care services, with individual identifiers separated by a separator (e.g., a comma or pipe). Each observation in Table AAA7 may also include a unique identifier (hereafter referred to as AAA7-identifier).

### Example Table AAA0

A table, referred to as “Table AAA0,” for example, may be developed, which may house, at the observation level, metadata on all data sources from which data are used to develop Tables AAA1-AAA7. Each observation in this table may include information on different data sources from which data are used for any of the other tables described herein. Observation level data elements may include (but may not be limited to) a description of the original data source, time period (e.g., month and year) of the data, number of observations in the original data, number of observations extracted from the original data into selected data sets described herein, weighting variables and potential weighting schemes available in the original data, weighting variable and weighting schemes used to develop selected other data sets described herein. Each observation in Table AAA0 may have a unique identifier that is also present in observations in other data sets described herein, which contain data from the data source represented by the observation. Table AAA0 may be stored in a data repository and updated quarterly based on the most recently available data.

During the development of Tables AAA0 through AAA7, data may be extracted from source data sets and transmitted into target data sets electronically (e.g., using R, SAS, SQL, and/or Python-based programming). In some embodiments, Table AAA3 may be limited to between 100 million to 300 million observations to facilitate easier and faster computation using the data. This may be done while concurrently ensuring that a sufficient number of the observations with data on the relevant factors potentially associated with a patient's health condition are included in the data.

### Data Tables Storing Predictive Values Of Clinical Findings And Other Explanatory Factor Codes For Diseases And Disease Conditions

In some examples, a set of tables may be created and/or developed, identified herein for example purposes as Tables BBB0, BBB1, and BBB2, wherein the information regarding the associations between each clinical finding in the constellation of patient-reported problems, clinical exam findings, laboratory test results, imaging, and other diagnostic procedure findings, and family-behavioral-sociodemographic-medical history based risk factors and each diagnosis of a disease or disease condition are stored. The constellation of clinical findings used for the development of Tables BBB0 to BBB3 may be all those for which there are observations in Table AAA-2 that indicate that the code represented in the observation is a clinical finding. The constellation of diagnoses, and their codes (e.g., ICD-10-CM codes), used for the development of Tables BBB0 to BBB3 may be all those for which there are observations in Table AAA-2 that indicate the is a diagnosis of a disease or disease condition. For computational expedience, Tables BBB0 to BBB3 may be stored as partitioned tables, and other data structuring and processing expediencies may be applied as needed. Some or all tables may be stored in a cloud-based storage container.

### Example Table BBB0

A table referred to as “Table BBB0,” for example, may be developed wherein each observation in the table includes a unique combination of four data elements: the sex of the patient, the age group of the patient (e.g., 0-12 years, 13-20 years, 21-49 years, 50-64 years, >65 years), an AAA4-identifier based clinical finding, and a coded (e.g., ICD-10-CM coded) disease or disease condition. For each observation in Table BBB0, a variable holding a predictive parameter (Pred_Value_1) may be developed that reflects the association between the AAA4-identifier-based clinical finding and the ICD-10-CM diagnosis in each respective sex and age group. The process of developing Pred_Value_1, which includes the development and use of parameters ia, iia, iiia, and iva, is described herein.

Using data from Table AAA3 and descriptive and modeling analyses (e.g., using R and/or SAS statistical software and procedures such as proc logistic and glm function), the following relationships may be parameterized: parameter ia—how strongly an abnormal clinical finding predicts the diagnosis; parameter iia—how strongly a normal finding predicts the lack of the disease; parameter iiia—to what degree the abnormal finding predicts multiple diseases or disease conditions; and parameter iva—how commonly is the diagnosis seen among patients in the outpatient setting.

The parameter ia described herein may be derived through univariate logistic regression analyses where a separate univariate regression model may be run for each combination of sex, age group, abnormal clinical finding, and primary diagnosis that exists in Table AAA3. This may be done by first identifying all AAA4-identifier codes for all AAA1-identifier codes that exist in the abnormal clinical finding variable (which is a multi-data-element variable) in Table AAA3; the AAA1-identifier codes in Table AAA3 may be mapped to the respective AAA4-identifier code in Table AAA4 for this purpose. Then for each of these AAA4-identifiers, a separate univariate regression model may be run using all the observations in Table AAA3, with each observation having a 0/1 variable indicating whether the AAA4-identifier is present or not in the abnormal clinical finding variable. In some embodiments, this may result in a separate regression model being run for each pair or abnormal finding and diagnosis within each sex and age group (e.g., 0-12 years, 13-20 years, 21-49 years, 50-64 years, >65 years) combination with each iteration using the data from Table AAA3. To increase the efficiency of the process while maintaining accuracy, consideration may be given during application development and tuning for using a random sample drawn from Table AAA3 for generating parameter ia; the CI bounds for the resultant exp(beta) [exponentiated beta] values may be assessed, to determine whether to use random samples of Table AAA3 and the sample sizes to use. The form of the regression model for generating the parameter ia may be:

$$diagnosis ( presence ) = intercept + beta ( ia ) * ( AAA 4 - identifier present ) + / - error$$

The measurement of the association may be parameterized by the magnitude of the exp(beta) value from the regression model. Where the exp(beta) value is greater than 1, it may be entered into a new table, Table BBB1, which is further described herein. Specifically, the exp(beta) value may be entered into Table BBB1's cell for the respective sex, age group, AAA4-identifier-based abnormal clinical finding, and ICD-10-CM-based diagnosis combination as the value for parameter ia. Where the beta value is negative or non-derivable (e.g., because of lack of sufficient observations), the value for parameter ia may be set to 0 in the applicable cell in Table BBB1. During the application development and tuning process, further consideration may be given to restricting the eligibility of accepting positive beta values (and not setting parameter (ia) to zero) to those where the model includes at least a certain number of observations (e.g., 10,000) with the clinical finding and/or the diagnosis, where the beta value is statistically significant, and/or where the lower confidence bound lies a certain distance difference between 1 and the point estimate of the exp(beta) (e.g., at least one-quarter of the distance from 1). During the application development and tuning process, further consideration may also be given to setting an upper bound to the positive exp(beta) value (e.g., at 25).

The parameter iia described herein may be derived through univariate logistic regression analyses where a separate univariate regression model may be run for each combination of sex, age group, AAA4-identifier-based normal clinical finding, and primary diagnosis that exists in Table AAA3. This may be done by first identifying all AAA4-identifier codes for all AAA1-identifier codes that exist in the normal clinical finding variable (which is a multi-data-element variable) in Table AAA3; the AAA1-identifier codes in Table AAA3 may be mapped to the respective AAA4-identifier code in Table AAA4 for this purpose. Then for each of these AAA4-identifiers, a regression model may be run using all the observations in Table AAA3, with each observation having a 0/1 variable indicating whether the AAA4-identifier is present or not in the normal clinical finding variable. In some embodiments, this may result in a separate regression model being run for each pair or normal finding and diagnosis within each sex and age group (e.g., 0-12 years, 13-20 years, 21-49 years, 50-64 years, >65 years) combination with each iteration using the data from Table AAA3. To increase the efficiency of the process while maintaining accuracy, consideration may be given during application development and tuning for using a random sample drawn from Table AAA3 for generating parameter ia; the CI bounds for the resultant exp(beta) [exponentiated beta] values may be assessed, to determine whether to use random samples of Table AAA3 and the sample sizes to use. The form of the regression model for generating parameter iia may be:

$$diagnosis ( absence ) = intercept + beta ( iia ) * ( AAA 4 - identifier present ) + / - error$$

The measurement of the association may be parameterized by the negative value (i.e., multiplying by −1) of the magnitude of the exp(beta) value from the regression model. Where the exp(beta) value is greater than 1, its negative may be entered into Table BBB1's cell for the respective sex, age group, AAA4-identifier-based normal clinical finding, and primary diagnosis combination as the value for parameter iia. Where the beta value is negative or non-derivable (e.g., because of a lack of sufficient observations), the value for parameter iia may be set to 0 in the applicable cell in Table BBB1. During the application development and tuning process, further consideration may be given to restricting the eligibility of accepting negative beta values (and not setting parameter iia to zero) to those where the model includes at least a certain number of observations (e.g., 10,000) with the clinical finding and/or the diagnosis, where the beta value is statistically significant, and/or where the lower confidence bound lies a certain distance difference between 1 and the point estimate of the exp(beta) (e.g., at least one-quarter of the distance from 1). During the application development and tuning process, further consideration may also be given to setting a lower bound to the negative exp(beta) value (e.g., at −25).

The parameter iiia may be derived by first enumerating for each AAA4-identifier-based abnormal clinical finding within each sex and age group (e.g., 0-12 years, 13-20 years, 21-49 years, 50-64 years, >65 years) combination the number of primary diagnoses for which it has a positive parameter (ia). The inverse of this value may then be taken and scaled to a specified range (e.g., 1 to 3); the optimum scaling range may be determined during application development and tuning. An example of Table BBB0 is shown below.

Example Table BBB0

| Finding_{—} | Data_{—} | | | | Finding_{—} | Finding_{—} | Finding_{—} | Diagnosis_{—} | Diagnosis_{—} | Pred_{—} |
|---|---|---|---|---|---|---|---|---|---|---|
| Type | Source | Data_Year | Gender | Age-Grp | Type | Code | Description | Code | Description | Value_A |
| ABN | ABCD_1 | 2018_2019 | F | 15to | Symptom | RFV24150 | Glaucoma | Z961 | Presence of | 51.24 |
| | | | | 64 | | | | | intraocular | |
| | | | | | | | | | lens | |
| ABN | ABCD_1 | 2018_2019 | F | 15to | Symptom | RFV18450 | Symptoms | L821 | Other | 25.73 |
| | | | | 64 | | | of skin | | seborrheic | |
| | | | | | | | moles | | keratosis | |
| ABN | ABCD_1 | 2018_2019 | M | Under_15 | Symptom | RFV13551 | Earache, | J069 | Acute upper | 4.67 |
| | | | | | | | pain | | respiratory | |
| | | | | | | | | | infection, | |
| | | | | | | | | | unspecified | |
| ABN | ABCD_1 | 2018_2019 | M | 15to | Symptom | RFV46050 | Medical | D229 | Melanocytic | 1.64 |
| | | | | 64 | | | counseling, | | nevi, | |
| | | | | | | | NOS | | unspecified | |
| ABN | ABCD_1 | 2018_2019 | F | 15to | Symptom | RFV11351 | Insomnia | F429 | Obsessive- | 9.69 |
| | | | | 64 | | | | | compulsive | |
| | | | | | | | | | disorder, | |
| | | | | | | | | | unspecified | |
| ABN | ABCD_1 | 2018_2019 | F | 15to | Symptom | RFV27350 | Diagnosed | O368 | Maternal | 5.65 |
| | | | | 64 | | | complications | | care for other | |
| | | | | | | | of | | specified | |
| | | | | | | | pregnancy | | fetal | |
| | | | | | | | | | problems | |
| ABN | ABCD_1 | 2018_2019 | M | 15to | Symptom | RFV23050 | Functional | F840 | Autistic | 25.55 |
| | | | | 64 | | | psychoses | | disorder | |
| ABN | ABCD_1 | 2018_2019 | M | 15to | Symptom | RFV32300 | Eye | ZZZ0 | NA | 8.21 |
| | | | | 64 | | | examination | | | |
| ABN | ABCD_1 | 2018_2019 | F | 15to | Symptom | RFV67000 | For other | O368 | Maternal | 7.06 |
| | | | | 64 | | | and | | care for other | |
| | | | | | | | unspecified | | specified | |
| | | | | | | | test | | fetal | |
| | | | | | | | results | | problems | |
| ABN | ABCD_1 | 2018_2019 | M | 15to | Symptom | RFV22050 | Diabetes | H524 | Presbyopia | 6.18 |
| | | | | 64 | | | mellitus | | | |
| ABN | ABCD_1 | 2018_2019 | M | 15to | Symptom | RFV19400 | Shoulder | S460 | Injury of | 53.59 |
| | | | | 64 | | | symptoms | | musc/tend | |
| | | | | | | | | | the rotator | |
| | | | | | | | | | cuff of | |
| | | | | | | | | | shoulder | |
| ABN | ABCD_1 | 2018_2019 | F | 15to | Symptom | RFV64000 | For | S525 | Fracture of | 26.29 |
| | | | | 64 | | | radiological | | lower end of | |
| | | | | | | | findings | | radius | |
| ABN | ABCD_1 | 2018_2019 | M | 15to | Symptom | RFV48000 | Progress | M109 | Gout, | 7.19 |
| | | | | 64 | | | visit, NOS | | unspecified | |
| ABN | ABCD_1 | 2018_2019 | F | 15to | Symptom | RFV45600 | Other | G35- | NA | 3.06 |
| | | | | 64 | | | specific | | | |
| | | | | | | | therapeutic | | | |
| | | | | | | | procedures | | | |
| ABN | ABCD_1 | 2018_2019 | F | 65n | Symptom | RFV29000 | Arthritis | M255 | Pain in joint | 5.42 |
| | | | | Older | | | | | | |
| ABN | ABCD_1 | 2018_2019 | F | 15to | Symptom | RFV14551 | Throat | J069 | Acute upper | 45.04 |
| | | | | 64 | | | soreness | | respiratory | |
| | | | | | | | | | infection, | |
| | | | | | | | | | unspecified | |
| ABN | ABCD_1 | 2018_2019 | F | 65n | Symptom | RFV28150 | Eczema | Z858 | Personal | 1.49 |
| | | | | Older | | | and | | history of | |
| | | | | | | | dermatitis, | | malig | |
| | | | | | | | NOS | | neoplasms of | |
| | | | | | | | | | organs and | |
| | | | | | | | | | systems | |
| ABN | ABCD_1 | 2018_2019 | F | 65n | Symptom | RFV29100 | Other | C449 | Other and | 30.91 |
| | | | | Older | | | musculosk | | unsp | |
| | | | | | | | eletal or | | malignant | |
| | | | | | | | connective | | neoplasm of | |
| | | | | | | | t . . . | | skin, | |
| | | | | | | | | | unspecified | |
| ABN | ABCD_1 | 2018_2019 | M | 65n | Symptom | RFV27 | Urinary | Z018 | Encounter | 1.64 |
| | | | | Older | | 050 | tract | | for other | |
| | | | | | | | disease | | specified | |
| | | | | | | | except | | special | |
| | | | | | | | cystitis | | examinations | |
| ABN | ABCD_1 | 2018_2019 | F | 15to | Symptom | RFV12050 | Convulsions | E119 | Type 2 | 2.22 |
| | | | | 64 | | | | | diabetes | |
| | | | | | | | | | mellitus | |
| | | | | | | | | | without | |
| | | | | | | | | | complications | |
| ABN | ABCD_1 | 2018_2019 | M | 15to | Symptom | RFV17153 | Growths, | N508 | Other | 64.49 |
| | | | | 64 | | | warts, | | specified | |
| | | | | | | | lumps, | | disorders of | |
| | | | | | | | bumps of | | male genital | |
| | | | | | | | the s . . . | | organs | |
| ABN | ABCD_1 | 2018_2019 | M | 15to | Symptom | RFV10200 | General | I10- | NA | 2.23 |
| | | | | 64 | | | weakness | | | |
| ABN | ABCD_1 | 2018_2019 | F | 65n | Symptom | RFV42050 | Postoperative | Z471 | Aftercare | 16.39 |
| | | | | Older | | | visit | | following | |
| | | | | | | | | | joint | |
| | | | | | | | | | replacement | |
| | | | | | | | | | surgery | |
| ABN | ABCD_1 | 2018_2019 | M | 65n | Symptom | RFV32300 | Eye | H353 | Degeneration | 4.87 |
| | | | | Older | | | examination | | of macula | |
| | | | | | | | | | and posterior | |
| | | | | | | | | | pole | |
| ABN | ABCD_1 | 2018_2019 | M | Under_15 | Symptom | RFV42050 | Postoperative | Q899 | Congenital | 57.87 |
| | | | | | | | visit | | malformation, | |
| | | | | | | | | | unspecified | |
| ABN | ABCD_1 | 2018_2019 | F | 15to | Symptom | RFV10150 | Tiredness, | T833 | Mechanical | 86.88 |
| | | | | 64 | | | exhaustion | | complication | |
| | | | | | | | | | of | |
| | | | | | | | | | intrauterine | |
| | | | | | | | | | contraceptive | |
| | | | | | | | | | device | |
| ABN | ABCD_1 | 2018_2019 | M | 15to | Symptom | RFV48000 | Progress | C029 | Malignant | 12.39 |
| | | | | 64 | | | visit, NOS | | neoplasm of | |
| | | | | | | | | | tongue, | |
| | | | | | | | | | unspecified | |
| ABN | ABCD_1 | 2018_2019 | F | 15to | Symptom | RFV19001 | Neck pain, | Z018 | Encounter | 24.32 |
| | | | | 64 | | | ache, | | for other | |
| | | | | | | | soreness, | | specified | |
| | | | | | | | discomfort | | special | |
| | | | | | | | | | examinations | |
| ABN | ABCD_1 | 2018_2019 | M | 15to | Sympt | RFV23700 | Other and | M501 | Cervical disc | 30.22 |
| | | | | 64 | om | | unspecified | | disorder with | |
| | | | | | | | diseases | | radiculopathy | |
| | | | | | | | of the . . . | | | |

### Example Table BBB1

A table, referred to as “Table BBB1,” for example, may be developed that may contain an observation (row) for each sex, age group, AAA4-identifier based clinical finding, and primary diagnosis combination that pertains to parameter (ia) and parameter (iia); a variable (column) in the table may hold the diagnosis, another column may hold its associated code (e.g., ICD-10-CM code), and other variables may hold the sex and age group. The table may also have a separate variable (column) for each AAA4-identifier based normal and abnormal finding that pertains to parameter (ia) and parameter (iia) and a column indicating if it was an abnormal or normal finding. The cells of another column of the table may hold the parameter value (parameter ia or parameter iia, as applicable) for the respective sex, age-group, ICD-10-CM-based diagnosis and the AAA4-identifier-based abnormal/normal finding combination. Table BBB1 may be stored as a partitioned data set.

The comparative strengths of parameters ia, iia, and iiia in their predictive contribution towards identifying potential primary diagnoses for diseases and disease conditions across all such diagnoses or groupings of such diagnoses (i.e., not specific to a single diagnosis and parameter pair) may be determined through multivariable logistic regression modeling using a large random sample of observations from the pool of all observations that were used for the regression models used to derive parameters ia and iia. The process for this may include the following steps. A random sample (e.g., a random sample of 10,000 observations) may be drawn from Table BBB1, which may form Table AB1. The vector (Diagnosis_Vector_A) of all unique primary diagnoses, sex, and age group combinations that exist in Table AB1 may be developed from said table. The vector (Findings_Vector_B) of all AAA4-identifier-based abnormal and normal clinical findings that exist in Table AB1 may be developed from said table. Random samples—may be drawn from Table ABI to form Diagnosis_Vector_A and Findings_Vector_B.

A Table AB2 may be developed by filtering Table AB1 to exclude all columns holding AAA4-identifier-based clinical findings other than those in Findings_Vector_B. For each unique sex, age group and primary diagnosis combination (element (i)) in

Diagnosis_Vector_A, a new table may be formed by converting Table AB2 to AB2[i] (with [i] representing the unique sex, age group and primary diagnosis combination) so that the new table has a general_diagnosis variable that is set to 0/1 based on the presence or absence of the primary diagnosis in element (i). This table may then be first pivoted with respect to all abnormal findings variables; the resultant table may then be pivoted again with respect to all normal findings variables. After completion of this process (i.e., development of a double pivoted table for each sex, age group and primary diagnosis element in Diagnosis_Vector_A, all of the new AB2[i] tables may be vertically linked to form Table AB3. The parameter iiia that was developed in the steps described above may then be included as a variable (i.e., column) in Table AB3 by merging these values using the AAA4-identifier-based abnormal clinical finding's variable name, sex, and age group as the linking variables. The parameters iab and iiab may then be derived through a multiple linear regression model run using Table AB3. The form of the model may be:

$$general\_diagnosis ( 1 ) = intercept + beta 1 * ( parameter ia * parameter iiia ) + beta 2 * ( parameter iia ) + / - error$$

Using the results of the model, parameter iab may be set to equal beta 1 and parameter iiab may be set to equal beta2.

For each observation (i.e., each row holding a unique combination sex, age group, AAA4-identifier-based clinical finding, and primary diagnosis) in Table BBB0, the value of Pred_Value_1 may be set according to the following:

For observations with abnormal clinical findings:

$$Pred\_Value \_ 1 = parameter ia ( for the corresponding unique combination of sex , age group , AAA 4 - identifier based clinical finding , and ICD - 10 - CM based diagnosis ) * parameter iiia ( for the corresponding unique combination sex , age group , AAA 4 - identifier based clinical finding , and ICD - 10 - CM based diagnosis ) * parameter iab$$

For observations with normal clinical findings:

$$Pred\_Value \_ 1 = parameter iia ( for the corresponding unique combination of sex , age group , AAA 4 - identifier based clinical finding , and ICD - 10 - CM based diagnosis ) * parameter iiab$$

Table BBB0 may be filtered to exclude all observations where Pred_Value_1=0.

The parameter iva may be derived by enumerating the number of each ICD-10-CM-based primary diagnosis for a disease or disease condition that exists in Table AAA3. The parameter iva may be stored in Table BBB3 with a variable (column) holding the ICD-10-CM diagnosis code and a variable (column) holding the corresponding parameter iva.

To improve the operational efficiency of the application 135 when mapping clinical findings from user inputs about a patient's clinical findings to the corresponding Pred_Value_1s associated with ICD-10-CM-based diagnoses, consideration may be given to limiting Table BBB0 to only hold a certain number of observations (e.g., observations with the highest 3 million positive Pred_Value_1 values and the observations with the lowest 3 million Pred_Value_1 values.

Document Repositories and Data Tables Housing information on Diagnosis-Specific Disease Management Guidance and Diagnosis-Specific Information

In some examples, a set of tables may be created and/or developed, identified herein for example purposes as Tables CCC1 and CCC2, that may store informational material with respect to clinical guidance documents associated with various diseases or disease conditions as well as information on lab tests ordered, lab test results, diagnostic procedures, diagnostic procedure findings, therapeutic procedures, patient care services, medications, and comorbidities commonly seen associated with various ICD-10-CM based diagnoses. In addition, web links to high metrics (e.g., high Altmetric) peer-reviewed journal articles that are associated with various diagnoses may also be stored within these tables.

A repository of Clinical Practice (management) Guideline documents may be developed for all commonly seen diseases and disease conditions. Attempts may be made to ensure that there is at least one practice guidelines document available for each disease and/or disease group (e.g., each disease and/or disease group coded by the first three characters of the ICD-10-CM code (the alpha character and the first two numeric digits of the ICD-10 code)). Each clinical guidelines documents may include evidence-based information about the disease and disease condition, including disease description, epidemiology, causal factors, risk factors, symptoms, signs, laboratory findings, imaging findings, recommended workups and management options, prognostic makers, screening recommendations, preventive measures related recommendations, medication interaction guidance, and references for the information included. The clinical practice guidelines documents may be developed through the use of the current information in the medical literature (with appropriate citation and permissions, where applicable), including medical textbooks, journal publications, and web-based information from professional bodies and healthcare-related institutions. The documents may be stored in a cloud storage container, with each document identifiable by a unique identifier (the practice-document-identifier); all documents may be reviewed and updated periodically as necessary.

### Example Table CCC1

A table, referred to as “Table CCC1,” for example, may be developed wherein each observation includes a variable (column) holding a unique practice-document-identifier and another variable (column) holding the ICD-10-CM code with which the clinical practice document is associated.

### Example Table CCC2

A table referred to as “Table CCC2,” for example, may be developed wherein each observation may include a variable for a unique disease or disease condition and separate variables for the ten most common lab tests ordered, lab test results, imaging, imaging results, diagnostic procedures, diagnostic procedure findings, medications, therapeutic procedures, comorbidities, and patient care services that are associated with the diagnosis in that observation. Table CCC2 may include observations for all disease or disease condition housed in Table AAA3. Note that to be included as one of the ten most common lab tests ordered, lab test results, imaging, imaging results, diagnostic procedures, medications, therapeutic procedures, comorbidities, and patient care services, each of these items may need to meet a certain threshold on prevalence with regard to its association with the disease or disease condition code (e.g., present in at least 10% of observations seen in Table AAA3 for the respective disease or disease condition). If at least ten items do not meet the threshold, then only those meeting the threshold may be included in the respective observation. Table CCC2 may be developed by first creating an interim table (Table CCC2-interim) which may include all observations in Table AAA3. However, in Table CCC2-interim, each code for lab tests ordered, lab test results, imaging, imaging results, diagnostic procedures, medications, therapeutic procedures, comorbidities, and patient care services that appear in the multi-data-element variables housing AAA1-identifier codes for these in Table AAA3 may be mapped first to the corresponding observation (with the same AAA1-identifier) that exist in Table AAA4, Table AAA5, Table AAA6, or Table AAA-7 and thereafter, the corresponding AAA4-identifier, AAA5-identifier, AAA-6-identifier, or the AAA-7-identifier may be included in a multi-data-element variable for that information element (for clinical findings, lab tests ordered, lab test results, imaging, imaging results, diagnostic procedures, therapeutic procedures, medications, comorbidities, and services depending on what the code represents).

All observations in Table CCC2-interim may then be filtered for each diagnosis. For each diagnosis-based set of observations, the frequency of appearance of each AAA4-identifier, AAA5-identifier, AAA6-identifier, or AAA7-identifier-based lab tests ordered, lab test results, imaging, imaging results, diagnostic procedures, medications, therapeutic procedures, comorbidities, and patient care services may be determined, and the ten most frequent lab tests ordered, lab test results, imaging, imaging results, diagnostic procedures, therapeutic procedures, medications, comorbidities, and services associated with each ICD-10-CM diagnosis may then be added to the observation in Table CCC2 housing ICD-10-CM diagnosis. Each of the lab tests, ordered lab test results, imaging, imaging results, diagnostic procedures, therapeutic procedures, medications, comorbidities, and services may be added as a separate variable in Table CCC2 with the variable names indicating the frequency order of the element in its association with the respective diagnosis (e.g., variables named LabTest1, LabTest2, LabTest3, LabTest4 LabTest10 from the most frequent to the 10th most frequent lab test associated with the ICD-10-CM diagnosis). For each of these variables (i.e., for the lab tests, lab test results, imaging, etc.), Table CCC2 may include corresponding variables holding descriptions for each coded element that may be drawn from Table AAA2.

Thereafter, the text description of the each diagnosis may be used in the search field of PubMed, and the titles, citations, and PubMed-abstract-weblinks of the ten journal articles with the highest metrics (e.g., highest Altmetrics) may be copied into separate variables for each of these in the respective observation in Table CCC2. Table CCC2 may be stored in a cloud storage container and updated periodically as necessary. An example of Table CCC2 is shown below.

Example of Table CCC2

| | | | Imagings_{—} | | | |
|---|---|---|---|---|---|---|
| Diagnosis_{—} | Examinations_{—} | Relative_{—} | and | Relative_{—} | Medications_{—} | Relative_{—} |
| Code | and_Labs_a | Frequency_a | Procedures_b | Frequency_b | Prescribed_c | Frequency_c |
| Z858 | Comprehensive | 4 | Cryosurgery | 32 | HYDROCHLOROTHIAZIDE | 25 |
| | metabolic | | (cryotherapy)/ | | | |
| | panel (CMP) | | Destruction of | | | |
| | | | tissue | | | |
| C61- | Comprehensive | 17 | Other imaging | 2 | MULTIVITAMIN | 25 |
| | metabolic | | | | | |
| | panel (CMP) | | | | | |
| H669 | Culture, other | 1 | CT Scan | 1 | CIPRO | 10 |
| I495 | CBC | 2 | Electromyogram | 2 | ASPIRIN | 20 |
| | (Complete | | (EMG) | | | |
| | blood count) | | | | | |
| M859 | Neurologic | 4 | X-ray | 4 | STIMATE | 6 |
| | exam | | | | | |
| M480 | Foot exam | 2 | CT Scan | 2 | IBUPROFEN | 25 |
| G409 | HgbA1C | 2 | MRI | 2 | KEPPRA | 15 |
| | (Glycohemoglobin | | | | | |
| L720 | Skin exam | 45 | Biopsy | 11 | BETADINE | 16 |
| J302 | Skin exam | 2 | Audiometry | 2 | METFORMIN | 1 |
| | | | | | HYDROCHLORIDE | |
| | | | | | ER | |
| I272 | CBC | 57 | EKG/ECG | 19 | ALBUTEROL | 8 |
| | (Complete | | | | | |
| | blood count) | | | | | |
| Z858 | Skin exam | 51 | CT Scan | 4 | GLIPIZIDE | 25 |
| I15- | Neurologic | 7 | EKG/ECG | 7 | VIMPAT | 4 |
| | exam | | | | | |
| H251 | HgbA1C | 11 | Tonometry | 14 | MOXIFLOXACIN | 5 |
| | (Glycohemoglo | | | | | |
| | bin) | | | | | |
| G35- | Comprehensive | 6 | MRI | 15 | SERTRALINE | 5 |
| | metabolic | | | | | |
| | panel (CMP) | | | | | |
| E039 | Lipid profile | 4 | Bone mineral | 9 | NATURE- | 10 |
| | | | density | | THROID | |
| H81- | Comprehensive | 21 | Other ultrasound | 12 | VITAMIN D3 | 9 |
| | metabolic | | | | | |
| | panel (CMP) | | | | | |
| D489 | Comprehensive | 2 | Biopsy | 12 | METOPROLOL | 16 |
| | metabolic | | | | SUCCINATE | |
| | panel (CMP) | | | | | |
| N433 | Rectal exam | 2 | Other ultrasound | 4 | LEVOTHYROXINE | 10 |
| Z858 | Skin exam | 51 | Cryosurgery | 32 | HYDROCHLOROTHIAZIDE | 25 |
| | | | (cryotherapy)/ | | | |
| | | | Destruction of | | | |
| | | | tissue | | | |
| L98- | Skin exam | 6 | Excision of | 6 | BIOTIN | 9 |
| | | | tissue | | | |
| I739 | Glucose, serum | 4 | CT Scan | 2 | TRIAMCINOLONE | 25 |
| | | | | | ACETONIDE | |
| J449 | CBC | 43 | CT Scan | 6 | CHANTIX | 25 |
| | (Complete | | | | | |
| | blood count) | | | | | |
| H353 | Lipid profile | 6 | Other ultrasound | 26 | AMLODIPINE | 20 |
| L659 | Basic | 13 | Bone mineral | 13 | BIOTIN | 5 |
| | metabolic | | density | | | |
| | panel ((BMP) | | | | | |
| S832 | Foot exam | 2 | MRI | 33 | ATENOLOL | 4 |
| M489 | Comprehensive | 41 | CT Scan | 2 | AMITRIPTYLINE | 10 |
| | metabolic | | | | | |
| | panel (CMP) | | | | | |
| Z000 | Breast exam | 41 | Other ultrasound | 37 | VITAMIN D3 | 25 |
| N201 | Culture, urine | 1 | CT Scan | 1 | AMITRIPTYLINE | 9 |
| Z038 | Comprehensive | 3 | Audiometry | 1 | LIPITOR | 15 |
| | metabolic | | | | | |
| | panel (CMP) | | | | | |

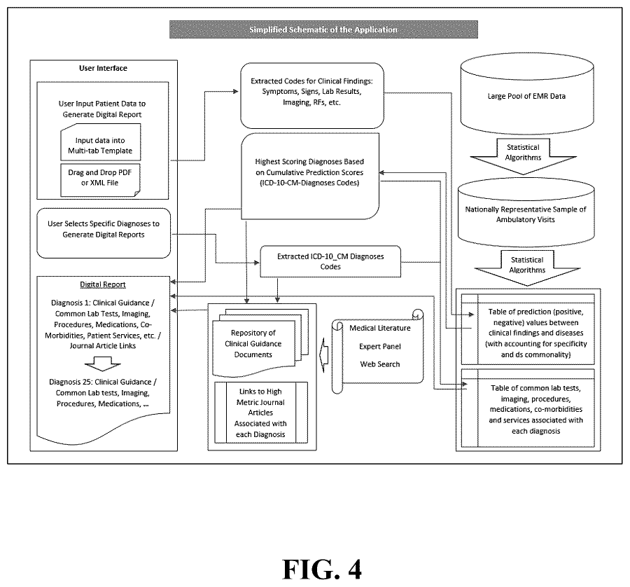

FIG. 2 shows a flow diagram of a process 200 for recommending potential diagnoses. In some embodiments, the process 200 can be performed by the system 100, including one or more processors 120 of the system 100. In some embodiments, the process 200 can be performed during execution of the application 135. FIG. 4 shows an example schematic of the application 135.

### User Interface Allowing Users (e.g., Clinician) To Generate Digital Reports

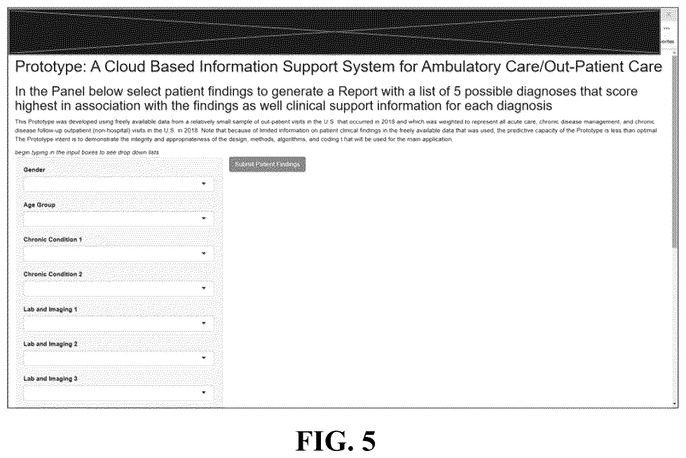

Referring to FIG. 2, in some embodiments, the process 200 can comprise causing 202 a user interface to be presented. A user may be able to input information about a patient (including e.g., patient demographics and clinical findings). Some examples of a user interface are shown in FIGS. 5-20.

After a user submits the information, the process 200 can further comprise receiving 206 user input indicating the clinical findings. As detailed hereafter, the user input can be used to generate digital reports that include Possible Diagnoses, Clinical Guidance Material Associated with each of the Diagnoses, and information on Lab Tests and Lab Test results, Imaging and Imaging Results, Diagnostic Procedures, Medications, Therapeutic Procedures, Comorbidities, and Patient Care Services commonly seen among patients with each of the respective diagnoses in a large sample of similar type of healthcare visits, e.g. a large sample primary care visits (which may be a nationally representative sample if primary care visits). The reports may also include links to high-metric peer-reviewed journal articles associated with each diagnosis.

A user may also be able to generate the report and information mentioned above directly for a selected diagnosis (as chosen by the user) without needing to input any patient information.

### Web-based User Interface Allowing Users To Log In And Begin Using the Application

In some embodiments, in the application 135, an initial user interface page may be presented where users (e.g., Clinicians) granted access to the application 135 may be able to log in using their access verification information. This page may provide information about the application 135, conditions related to its use, and what the application 135 does and does not provide. The page may also provide at least the following two option buttons for the user to choose for the next steps in using the application: 1) A button that may take the user to a page where patient information can be inputted and used to generate digital reports that include Possible Diagnoses, Clinical Guidance Material associated with each of the Diagnoses, information on Lab Tests, Lab Test Results, Imaging, Imaging Results, Diagnostic Procedure, Medications, Therapeutic procedures, Comorbidities, and Patient Care Services commonly seen among patients with each of the same Diagnoses in a large Nationally Representative Sample of Outpatient Visits as well as links to high metric peer-reviewed journal articles associated with each diagnosis and 2) A button that may take the user to a page where the user can select one or more diagnoses (up to e.g., 10) and generate digital reports for each diagnosis that includes all of the information described above for option number 1.

### Web-Based User Interface Component Allowing Users to Input Patient Information to Generate Digital Reports

In some embodiments, in the application 135, a multi-tab interactive template is provided for inputting information about patient demographics, clinical findings (including patient-reported symptoms, clinical examination findings, lab results, imaging findings, chronic conditions, behavioral risk factors, and family medical history). Upon submitting the information, the information may be received and processed to generate a digital report.

A user may have two options by which the patient information can be inputted. The two options may not be mutually exclusive, and the user may be able to use both options for inputting patient-related information. The two options are described below.

Option 1—Manually enter the information in interactive information fields that provide drop-down choices (where selection suggestions are provided based on typing in partial text related to the information element). The interactive information fields may be organized in separate tabs by type of information element, such as, for example, patient demographics, clinical findings, lab results, imaging findings, chronic conditions history, behavioral risk factors, family medical history, and other information.

Option 2—Upload (or drag and drop) a file (e.g., a pdf or an XML file) that includes patient information, including patient demographics, clinical findings, lab results, imaging findings, chronic conditions history, behavioral risk factors, family medical history, and other information. This may be a file (e.g., a pdf or XML file) that is generated by the user's own office or practice-based patient care-related software (e.g., the clinician's electronic medical records [EMR] software.

Upon selecting Option-1, the user may be directed to a multi-tab page where there may be a tab for each of the following: patient demographics, clinical findings, lab results, imaging findings, chronic conditions history, behavioral risk factors, family medical history, and other information. The Patient Demographics tab may have information fields for selecting Patient sex, age group, race, and ethnicity from drop-down categories to select from. The Clinical Findings, Lab Results, Imaging Findings, Chronic Conditions History, Behavioral Risk Factors-Family Medical History, and Other Findings tabs each may have ten information fields. In each information field, the user may be able to select from a list of choices that may appear when the user clicks in the field; upon entering partial text into the field, the drop-down list may be shortened to match choices that match the partial text. Note that the sex and age group of the patient may be fields where information may be required. The choices available in the information fields may be all of the items (e.g., clinical findings, lab test results, imaging results) that exist in Table AAA2. The choices displayed for the provider may include both the code (e.g., SNOMED, LOINC, ICD-10-CM code) associated with the information item as well as the description of the information item (e.g., the clinical finding or lab test result). Below the ten information fields on each page, there may be an option to add additional information fields, which may function the same way as the ten fields already in each tab. The user may input information in 0 to 10 or more information fields in each tab.

Upon selecting Option 2, the user may be directed to the same multi-tab page for inputting patient information as with Option 1. However, on this page, there may also be a window opened where the user may be able to upload (or drag and drop) a file (e.g., a pdf or XML file) containing the patient's information. After the file is uploaded by the user, the file may be searched using natural language processing programs (e.g., using R or Python coding) to identify all data elements (e.g., codes or descriptions very likely matching any of the data elements in the lists associated with the information fields in the tabs for Patient Demographics, Clinical Findings, Lab Results, Imaging Findings, Chronic Conditions History, Behavioral Risk Factors-Family Medical History, and Other Findings (note that the lists are drawn from Table AAA2). In this process, text-matching techniques such as string matching, fuzzy-matching, Jaro-Winkler, and Named Entity Recognition-based algorithms may be used to match the information in the uploaded file to the item codes and item descriptions in Table AAA2. All information elements in Table AAA2 that are matched may then be populated into information fields in the applicable tab of the multi-tab page for inputting patient information.

At the top of each tab, the user may be prompted to review the information input in the tabs and confirm that these are the inputs to use to generate the report. The user may also be informed that the user can change any of the inputs (whether manually entered or populated automatically via uploading of a file) as needed. In addition, the user may be informed that once the user is satisfied with the inputted information, to go to the last tab where the user can press the Submit button to generate the report.

After the user has confirmed that all the inputs on the page would be the information used to generate a report, he/she presses the Submit button.

The information may be used to generate a Digital Report that includes Possible Diagnoses, Clinical Guidance Material associated with each of the Diagnoses, and information on Lab tests, Lab Test Results, Imaging, Imaging Results, Diagnostic Procedure, Medications, Therapeutic procedures, Comorbidities, and Patient Care Services commonly seen among patients with each of the same Diagnoses in a large sample (e.g. a nationally representative sample) of primary care visits as well as links to high metric peer-reviewed journal articles associated with each diagnosis. This may be done through the processes outlined herein.

### Web-based User Interface Component Allowing Users to Select Disease and Disease Conditions

In the application 135, a web page may be developed where the user may be able to select diseases and disease conditions (up to 10) and submit these to generate digital reports with clinical support information for each diagnosis.

A web page may be developed with ten information fields where the user can choose from a drop-down list diagnoses for diseases and disease conditions (along with their codes, e.g. ICD-10-CM codes) in each field. The list may include all diagnoses for diseases and disease conditions that are included in Table AAA2. In each information field, the user may be able to select from a list of choices that may appear based on the partial text the user puts into the field.

After selecting one or more diseases or disease conditions, the user may be able to submit these by pressing the Submit button to generate a digital report that includes Possible Diagnoses, Clinical Guidance Material associated with each of the Diagnoses, and information on Lab Tests, Lab Test results, Imaging, Imaging Results, Diagnostic Procedure, Medications, Therapeutic procedures, Comorbidities, and Patient Care Services commonly seen among patients with each of the same Diagnoses in a large Nationally Representative Sample of Outpatient Visits as well as links to high metric peer-reviewed journal articles associated with each diagnosis. This may be done through the processes outlined herein.

### Generation of Possible Diagnoses Based on Inputs About the Patient and Feedback to Users via Digital Reports

After receiving 206 the user input, the process 200 can further comprise determining 210 clinical codes corresponding to the clinical findings. In some embodiments, the clinical codes are any clinical codes included in any table, such as, for example, Tables AAA2 and/or AAA4.

In some embodiments, the process 200 can further comprise determining 214 that one or more first clinical findings are normal clinical findings, and determining 214 that one or more second clinical findings are abnormal clinical findings. In some embodiments, Table AAA2 can be used to determine which clinical findings are normal clinical findings and which clinical findings are abnormal clinical findings.

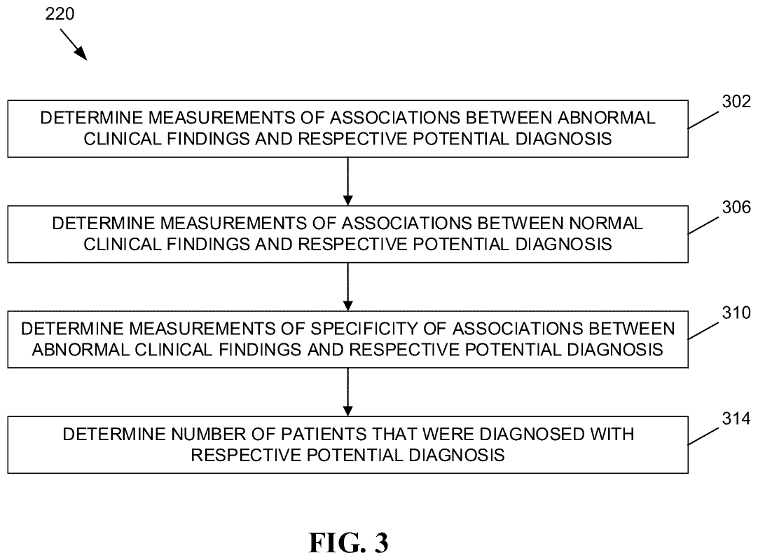

In some embodiments, the process 200 can further comprise determining 220 a plurality of diagnostic scores for respective potential diagnoses of a plurality of potential diagnoses. Referring to FIG. 3, determining 220 a diagnostic score for a respective potential diagnosis of the plurality of potential diagnoses can comprise determining 302 one or more measurements of associations between the abnormal clinical findings and the respective potential diagnosis. In some embodiments, the one or more measurements of associations between the abnormal clinical findings and the respective potential diagnosis can include one or more parameters ia described herein.

In some embodiments, the process 220 can include determining 306 one or more measurements of inverse associations between the normal clinical findings and the respective potential diagnosis. In some embodiments, the one or more measurements of inverse associations between the normal clinical findings and the respective potential diagnosis can include one or more parameters iia described herein.

In some embodiments, the process 220 can include determining 310 one or more measurements of specificity of associations between the abnormal clinical findings and the respective potential diagnosis. In some embodiments, the one or more measurements of specificity of associations between the abnormal clinical findings and the respective potential diagnosis can include one or more parameters iiia described herein. A measurement of specificity of an association between an abnormal finding and a potential diagnosis can be based in part on a number of measurements of associations between the abnormal finding and potential diagnoses that are above a predetermined threshold. A larger number of measurements of associations between the abnormal finding and potential diagnoses that are above the predetermined threshold can result in a lower measurement of specificity of association between the abnormal finding and any potential diagnosis.

In some embodiments, the process 220 can include determining 314 a number of patients that were diagnosed with the respective potential diagnosis during one or more periods of time. In some embodiments the process 220 can further comprise determining a measurement of a commonality of the respective potential diagnosis, wherein the measurement of the commonality of the respective diagnosis is based on the number of patients that were diagnosed with the respective potential diagnosis during one or more periods of time. In some embodiments, the measurement of the commonality of the respective diagnosis can include the parameter iva described herein.

Referring to FIG. 2, after determining 220 the plurality of diagnostic scores, the process 200 can further comprise ranking 224 at least a portion of the plurality of potential diagnoses based at least on the plurality of diagnostic scores. In some embodiments, ranking a potential diagnose higher indicates that the patient is more likely to be diagnosed with the potential diagnosis based on the user input. Based on the inputs by a user (e.g., clinician) in the user interface's patient information input tabs, all codes associated with patient problems, clinical findings, laboratory test results, imaging findings, diagnostic procedure results, family-behavioral-sociodemographic-medical history-based risk factors, etc. may be used to generate a set of possible diagnoses of disease or disease conditions ranked by scores representing the comparative likelihood of association of each diagnosis with the inputs. For this purpose, the processes outlined below may be followed.

After confirmation of the patient inputs by the user in the user interface, all AAA1-identifiers associated with (patient problems, clinical findings, laboratory test results, imaging findings, family-behavioral-sociodemographic-medical history-based risk factors, etc.) may be transferred as a column vector into a table (Table FFF1). The sex and age group of the patient may be added as separate columns (variables) to the table. In a separate column (variable) in Table FFF1, the AAA4-identifier from Table AAA4 that correspond to the AAA1-identifier in each observation of Table FFF1 may be merged into that observation in Table FFF1. Table FFF1 may then be filtered to unique observations by the AAA4-identifiers to create Table FFF2. Table BBB0 may then be filtered to all sex, age group and AAA4-identifier combinations that exist in the observations of Table FFF2 to create Table FFF3. The Pred_Value 1 values in the observations of Table FFF3 may then be summed across all observations with the same diagnosis to create a Summed_Pred_Value_1 for each diagnosis. A Table FFF4 may be created that includes all unique diagnoses from Table FFF3, with each observation holding a unique diagnosis, its description, and the Summed_Pred_Value_1 for that diagnosis. In a separate variable in Table FFF4, the parameter iva from Table BBB3 may be merged in with diagnosis being the linking variable. A new variable (Relative_Score) may then be created in Table FFF4 by multiplying Summed_Pred_Value_1 by parameter iva (i.e., Relative_Score=Summed_Pred_Value_1*parameter iva). Table FFF5 may then be created by filtering Table FFF4 to a selected number of observations (e.g., 25 observations) with the highest Relative Score. Each of the diagnoses in Table FFF5, along with its code (e.g., ICD-10-CM code) and the Relative_Score may then be generated as a separate line in the Digital Report. The diagnoses may be ordered in descending order of Relative_Scores.

In some embodiments, the process 200 can further comprise causing 228 at least the portion of the plurality of potential diagnoses to be presented based at least on the ranking. In some embodiments, at least the portion of the plurality of potential diagnoses can be presented in a user interface. The user interface can be presented at a display such as, for example, display 150 in FIG. 1, or any other display communicatively connected to the system 100 in FIG. 1.

In some embodiments, the process 200 can further comprise causing clinical guidance information associated with at least the portion of the potential diagnoses to be presented. In some embodiments, pieces of clinical guidance information associated with respective potential diagnoses can be ranked based on the plurality of diagnostic scores for the respective potential diagnoses. In some embodiments, the pieces of clinical guidance information can be presented based on the ranking of the pieces of clinical guidance information.

Under each diagnosis displayed in the Digitals Report, there may be a set of buttons for displaying the clinical guidance information such as, for example: lists of the 10 lab tests ordered, lab test results, imaging ordered, imaging results, diagnostic procedures, diagnostic procedure findings, therapeutic procedures, medications, comorbidities, and patient care services commonly seen among patients with the diagnosis in a large nationally representative sample of outpatient visits, and the titles, citations, and PubMed-abstract-weblinks to the ten journal articles with the highest metrics. Thus, under diagnosis displayed in the digitals report, there may be 12 buttons with a label on each button indicating what will be displayed upon pressing the button (e.g., “Press this button to display tab test orders commonly seen among outpatients with this diagnosis”).

### Displaying Digital Reports

Along with displaying (in descending order of Relative_Scores) the diagnoses with the highest Relative_Scores of association with the user's (e.g., clinician's) inputs about the patient, the digital report may also display under each diagnosis as a set of 12 buttons for displaying the clinical guidance information such as, for example: lists of the ten most common clinical findings, lab tests ordered, lab test results, imaging, imaging results, diagnostic procedures, diagnostic procedure findings, therapeutic procedures, medications, comorbidities, and patient care services commonly seen among patients with that diagnosis in a nationally representative sample of outpatient visits, as well as the titles, citations, and PubMed-abstract-weblinks to the ten journal articles with the highest metrics (e.g., highest Altmetrics). Pressing each button may result in a table displaying the item indicated by the button (e.g., the ten most common Lab Test Orders seen among patients with the diagnosis). The processes for these actions are outlined herein.

Under each of the diagnoses displayed in the digital report, there may be a button that, when pressed, may display the Clinical Guidance Document associated with that diagnosis. This may be done by first retrieving the practice-document-identifier associated with the diagnosis in Table CCC1 and then, using the practice-document-identifier, retrieving and displaying the Clinical Practice (management) Guideline document from the repository of those documents.

The following buttons may also appear under each diagnosis: Most Common Clinical Findings Associated With The Diagnosis; Most Common Lab Tests Ordered Associated With The Diagnosis, Most Common Lab Test Results Associated With The Diagnosis, Most Common Diagnostic Procedures Associated With The Diagnosis, Most Common Medications Associated With The Diagnosis, Most Common Therapeutic Procedures Associated With The Diagnosis, The Most Common Patient Care Services Associated with the Diagnosis, and recent High Metric Peer Reviewed Publication associated with the diagnosis. Pressing each of these buttons may result in displaying the ten most common items for that item type that are associated with the respective diagnosis among a large nationally representative sample of ambulatory outpatient visits. This may be done by mapping the diagnosis (with respect to the button pressed; the diagnosis under which the button exists) to the corresponding observation in Table CCC2 that holds that diagnosis and then retrieving from Table CCC2 and displaying in the Digital Report the ten items called for by that button (e.g., ten most common Lab Test Orders). The items within each item type may be displayed in order of their frequency of association with the diagnosis (i.e., in the order they are housed in the Table CCC2, LabTest1, LabTest2, LabTest3, LabTest4, etc.).

On a sidebar of the digital report, the user (e.g., clinician) may have the following buttons to do the following optionally:

1. Buttons to Save and Print the digital report;

2. A button that may enable changing one or more of the input values related to the patient's information (e.g., clinical findings, lab results, etc.). This may take the user back to the patient information input template populated with the original inputs, which can then be changed by the user as he or she wants. Once any information is changed, the user may be able to resubmit the information and generate a new report;

3. A button to start a fresh information input for a patient. This may take the user back to a blank patient information input template, which the user then can input new information and submit for generating a digital report;

### User Options to Separately Submit One or More Diagnoses

As described herein, in the application 135, there may be a page where a user may have the option to select diseases and disease conditions (e.g., up to 10) and submit these to generate a digital report with a clinical guidance document and clinical support information for each diagnosis. The process followed for effecting this may be the same process es described herein, except that in this case, the processes may be applied to the diagnoses selected by the user instead of a set possible diagnosis generated based on inputs about patient findings.

### Advantages

### Cost Efficiency of the Application

The embodiments described herein provide various advantages over existing technologies and methods.

In one example, cost-efficiency and time-efficiency are gained where applications herein are designed so that the individual instances of its use by a user do not result in large computer processing needs. Resource-intensive processes may be frontloaded in an application development phase (and during periodic, e.g., quarterly, reviewing an updating of application components). As a result, during use, the application may be able to quickly and cost-efficiently (without requiring excessive computing power and time) identify potential diagnoses and components of a digital report.

### Development Phase/Review and Upkeep Phase

During the development and or review/upkeep phase, the development of a nationally representative data set comprised of electronic medical records may be created, maintained, and/or updated. Such a data set may be based on data patient and clinical information from outpatient visits, for example.

Further, during a development and or review/upkeep phase, the development of predictive values (measurements of associations and specificity of associations of abnormal findings with potential diagnoses, rule-out values of normal findings for potential diagnoses, and commonality of potential diagnoses) that associate patient findings with potential diagnoses may be carried out. The predictive values may be stored in a relational table from where they may be accessed within the application for further processing via efficient algorithms. For example, algorithms may take inputs from patient clinical findings to rapidly identify a set of possible diagnoses. The diagnoses may be displayed in a digital report to a user and may be ordered by relative predictive scores.

Further, during a development and or review/upkeep phase, identification of the most common lab tests ordered lab test results, diagnostic procedures, diagnostic procedure findings, imaging, imaging results, medications prescribed, therapeutic procedures, and patient care services associated with each diagnosis in a large sample of healthcare visits may be carried out and associated information stored in the relational tables described herein.

Also stored in the relational tables may be links to high metric peer-reviewed journal articles. The most common lab tests ordered lab test results, diagnostic procedures, diagnostic procedure findings, imaging, imaging results, medications prescribed, therapeutic procedures, patient care services and journal article links may be displayed in association with each respective diagnosis in a digital report displayed to a user, for example.

Further, during a development and or review/upkeep phase, the development of clinical guidance material for various diagnoses may be carried out and associated information stored in the relational tables described herein. The clinical guidance material may be displayed in association with each respective diagnosis in a digital report displayed to a user, for example.

Further, during a development and or review/upkeep phase, development of a user-friendly user interface wherein a user may easily input patient information through inputting patient information in multi-tab based patient information templates and/or by uploading (or dragging and dropping) files (e.g., PDF or XML files) with patient information may be carried out. A user can then submit the information to generate a digital report with possible diagnoses and clinical support information (as described above) for each of the diagnoses.

Further, during a development and or review/upkeep phase, an option in a user interface wherein a user may select specific diagnoses and submit these to generate digital reports for each that include clinical support information (as described herein) may be created.

### Operational Phase

During an operational phase, user-inputted patient information (e.g., clinical findings, lab and imaging results, risk factors) may be mapped to the relational tables housing predictive values (measurements of associations and specificity of associations of abnormal findings with potential diagnoses, rule-out values of normal findings, and commonality of potential diagnoses). Novel programming algorithms may be used to generate predictive scores for various diagnoses. In one example, the highest-scoring diagnoses (e.g. 25 highest scoring diagnoses) may be identified within this group and may be displayed in a digital report.

For example, each of the diagnoses may be mapped to the clinical support information (as described herein) associated with each diagnosis in the relational tables and included in a digital report.

In some embodiments, during an operational phase, when a user chooses to input various diagnoses to generate clinical support information, the user-inputted diagnoses may be each mapped to the clinical support information (as described herein) associated with each diagnosis in preset tables and included in a digital report.

The descriptions of the various embodiments of the present invention have been presented for purposes of illustration, but they are not intended to be exhaustive or limited to the embodiments disclosed. Many modifications and variations will be apparent to those of ordinary skills in the art without departing from the scope and spirit of the described embodiments. The terminology used herein was chosen to best explain the principles of the embodiments, the practical application or technical improvement over technologies found in the marketplace, or to enable others of ordinary skills in the art to understand the embodiments disclosed herein.

The present invention may be embodied as a system, a method, and/or a computer program product. The computer program product may include a computer-readable storage medium (or media) having computer-readable program instructions stored thereon for causing a processor to carry out aspects of the present invention.

The medium may be an electronic, magnetic, optical, electromagnetic, infrared, or semiconductor system for a propagation medium. Examples of a computer-readable medium may include a semiconductor or solid-state memory, magnetic tape, a removable computer diskette, random-access memory (RAM), a read-only memory (ROM), a rigid magnetic disk, and an optical disk. Current examples of optical disks include compact disk-read-only memory (CD-ROM), compact disk-read/write (CD-R/W), DVD, and Blu-Ray-Disk.

The computer-readable storage medium can be a tangible device that can retain and store instructions for use by an instruction execution device. The computer-readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer-readable storage medium includes the following: a portable computer diskette, a hard disk, a random-access memory (RAM), a read-only memory (ROM), an erasable programmable read-only memory (EPROM or Flash memory), a static random access memory (SRAM), a portable compact disk read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (for example, light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer-readable program instructions from the network and forwards the computer-readable program instructions for storage in a computer-readable storage medium within the respective computing/processing device.

The computer-readable program instructions for performing processes of the present invention may be assembler instructions, instruction-set-architecture (ISA) instructions, machine instructions, machine-dependent instructions, microcode, firmware instructions, state-setting data, or either source code or object code written in any combination of one or more programming languages, including an object-oriented programming language such as Smalltalk, C++, JAVA, Python, R or the like, and conventional procedural programming languages, such as the “C” programming language or similar programming languages. The computer-readable program instructions may execute entirely on the user's computer, partly on the user's computer as a stand-alone software package, partly on the user's computer, and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry, including, for example, programmable logic circuitry, field-programmable gate arrays (FPGA), or programmable logic arrays (PLA), may execute the computer-readable program instructions by utilizing state information of the computer-readable program instructions to personalize the electronic circuitry, in order to perform aspects of the present invention.

Aspects of the present invention may be described herein with reference to flowchart illustrations and/or block diagrams of methods, apparatus (systems), and computer program products according to embodiments of the invention. It will be understood that each block of the flowchart illustrations and/or block diagrams and combinations of blocks in the flowchart illustrations and/or block diagrams can be implemented by computer-readable program instructions.

These computer-readable program instructions may be provided to a processor of a general-purpose computer, special-purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart and/or block diagram block or blocks.

The computer-readable program instructions may also be loaded onto a computer, other programmable data processing apparatuses, or another device to cause a series of operational steps to be performed on the computer, other programmable apparatus or other devices to produce a computer-implemented process, such that the instructions which execute on the computer, other programmable apparatuses, or another device implement the functions/acts specified in the flowchart and/or block diagram block or blocks.

The following description of variants is only illustrative of systems, components, elements, acts, media, and methods considered to be within the scope of the invention and are not in any way intended to limit such scope by what is specifically disclosed or not expressly set forth. The systems, components, elements, acts, media, and methods as described herein may be combined and rearranged other than as expressly described herein and are still considered to be within the scope of the invention.

According to variation 1, a system for recommending potential diagnoses can comprise memory; and one or more processors, wherein the one or more processors are configured at least to; receive user input indicating a plurality of clinical findings, the plurality of clinical findings comprising: one or more patient demographics; one or more patient reported symptoms; one or more clinical examination findings; one or more clinical laboratory results; and one or more clinical imaging findings; wherein the one or more processors are further configured at least to: determine that one or more first clinical findings of the plurality of clinical findings are normal clinical findings; determine that one or more second clinical findings of the plurality of clinical findings are abnormal clinical findings; determine a plurality of diagnostic scores for respective potential diagnoses of a plurality of potential diagnoses, wherein each diagnostic score for a respective potential diagnosis of the plurality of potential diagnoses is based at least on: one or more measurements of associations between the abnormal clinical findings and the respective potential diagnosis; one or more measurements of inverse associations between the normal clinical findings and the respective potential diagnosis; one or more measurements of specificity of associations between the abnormal clinical findings and the respective potential diagnosis; and a number of patients that were diagnosed with the respective potential diagnosis during one or more periods of time; and wherein the one or more processors are further configured at least to: rank at least a portion of the plurality of potential diagnoses based at least on the plurality of diagnostic scores.

Variation 2 can include a system as in variation 1, wherein the one or more processors are further configured to: cause at least the portion of the plurality of potential diagnoses to be presented based at least on the ranking.

Variation 3 can include a system as in variation 1, wherein the one or more processors are further configured to: cause pieces of clinical guidance information associated with respective potential diagnoses of at least the portion of the plurality of potential diagnoses to be presented.

Variation 4 can include a system as in variation 3, wherein the one or more processors are further configured to: rank the pieces of clinical guidance information based on respective measures or association of potential diagnoses with the pieces of clinical guidance information.

Variation 5 can include a system as in variation 3, wherein the pieces of clinical guidance information include one or more laboratory test recommendations and one or more imaging recommendations.

Variation 6 can include a system as in variation 3, wherein the pieces of clinical guidance information include one or more diagnostic procedure recommendations, one or more recommended medications, one or more recommended therapeutic procedures, and one or more co-morbidities.

Variation 7 can include a system as in variation 1, wherein each of the plurality of potential diagnoses corresponds to a respective disease code (e.g., International Classification of Diseases, 10th Revision, Clinical Modification (ICD-10-CM) diagnosis code).

According to variation 8, a method for recommending potential diagnoses can comprise receiving user input indicating a plurality of clinical findings, the plurality of clinical findings comprising: one or more patient demographics; one or more patient reported symptoms; one or more clinical examination findings; one or more clinical laboratory results; and one or more clinical imaging findings; wherein the method further comprises determining that one or more first clinical findings of the plurality of clinical findings are normal clinical findings; determining that one or more second clinical findings of the plurality of clinical findings are abnormal clinical findings; determining a plurality of diagnostic scores for respective potential diagnoses of a plurality of potential diagnoses, wherein each diagnostic score for a respective potential diagnosis of the plurality of potential diagnoses is based at least on: one or more measurements of associations between the abnormal clinical findings and the respective potential diagnosis; one or more measurements of inverse associations between the normal clinical findings and the respective potential diagnosis; one or more measurements of specificity of associations between the abnormal clinical findings and the respective potential diagnosis; and a number of patients that were diagnosed with the respective potential diagnosis during one or more periods of time; and wherein the method further comprises ranking at least a portion of the plurality of potential diagnoses based at least on the plurality of diagnostic scores.

Variation 9 can include a method as in variation 8, further comprising: causing at least the portion of the plurality of potential diagnoses to be presented based at least on the ranking.

Variation 10 can include a method as in variation 8, further comprising: causing pieces of clinical guidance information associated with respective potential diagnoses of at least the portion of the plurality of potential diagnoses to be presented.

Variation 11 can include a method as in variation 10, further comprising: ranking the pieces of clinical guidance information based on respective measures of association with potential diagnoses.

Variation 12 can include a method as in variation 10, wherein the pieces of clinical guidance information include one or more laboratory test recommendations and one or more imaging recommendations.

Variation 13 can include a method as in variation 10, wherein the pieces of clinical guidance information include one or more diagnostic procedure recommendations, one or more recommended medications, one or more recommended therapeutic procedures, and one or more co-morbidities.

Variation 14 can include a method as in variation 8, wherein each of the plurality of potential diagnoses corresponds to a respective code (e.g., International Classification of Diseases, 10th Revision, Clinical Modification (ICD-10-CM) diagnosis code).

According to variation 15, a non-transitory computer-readable medium can include one or more instructions that, when executed by one or more processors, cause the one or more processors to perform a method for recommending potential diagnoses, the method comprising receiving user input indicating a plurality of clinical findings, the plurality of clinical findings comprising: one or more patient demographics; one or more patient reported symptoms; one or more clinical examination findings; one or more clinical laboratory results; and one or more clinical imaging findings; wherein the method further comprises determining that one or more first clinical findings of the plurality of clinical findings are normal clinical findings; determining that one or more second clinical findings of the plurality of clinical findings are abnormal clinical findings; determining a plurality of diagnostic scores for respective potential diagnoses of a plurality of potential diagnoses, wherein each diagnostic score for a respective potential diagnosis of the plurality of potential diagnoses is based at least on: one or more measurements of associations between the abnormal clinical findings and the respective potential diagnosis; one or more measurements of inverse associations between the normal clinical findings and the respective potential diagnosis; one or more measurements of specificity of associations between the abnormal clinical findings and the respective potential diagnosis; and a number of patients that were diagnosed with the respective potential diagnosis during one or more periods of time; and wherein the method further comprises ranking at least a portion of the plurality of potential diagnoses based at least on the plurality of diagnostic scores.

Variation 16 can include a non-transitory computer-readable medium as in variation 15, wherein the method further comprises: causing at least the portion of the plurality of potential diagnoses to be presented based at least on the ranking.

Variation 17 can include a non-transitory computer-readable medium as in variation 15, wherein the method further comprises: causing pieces of clinical guidance information associated with respective potential diagnoses of at least the portion of the plurality of potential diagnoses to be presented.

Variation 18 can include a non-transitory computer-readable medium as in variation 17, wherein the method further comprises: ranking the pieces of clinical guidance information based on respective measures of association with potential diagnoses.

Variation 19 can include a non-transitory computer-readable medium as in variation 17, wherein the pieces of clinical guidance information include one or more laboratory test recommendations and one or more imaging recommendations.

Variation 20 can include a non-transitory computer-readable medium as in variation 17, wherein the pieces of clinical guidance information include one or more diagnostic procedure recommendations, one or more recommended medications, one or more recommended therapeutic procedures, and one or more co-morbidities.

Variations herein can generally relate to medical applications, systems, and methods. Some embodiments herein are directed to cloud-based applications for clinical decision support for patient care and other related information needs by users (e.g. clinicians). Some embodiments permit users to easily input patient information comprising one or more patient reported symptoms, one or more clinical examination findings, one or more lab results, one or more imaging findings, one or more chronic conditions, one or more behavioral risk factors (e.g., tobacco use, alcohol consumption, physical inactivity, etc), family medical history, patient demographics, or any combination thereof. In some embodiments, the inputted information is used for generating one or more digital report that will include a list of (e.g., 10 to 25) diagnoses that scored highest as per a novel scoring system and clinical guidance document/s providing patient management information, lists of the most common (as seen in a large sample of patient visits) lab tests ordered, lab test results, imaging ordered, imaging findings, diagnostic procedures, diagnostic procedure findings, medications prescribed, therapeutic procedures, co-morbidities, and patient care services associated with the diagnosis, as well as the titles, citations, and PubMed-abstract-weblinks to the 10 journal articles with the highest metrics.

The digital reports also include clinical guidance document/s providing patient management information, lists of the most common (as seen in a large sample of patient visits) lab tests ordered, lab test results, imaging ordered, imaging findings, diagnostic procedures, diagnostic procedure findings, medications prescribed, therapeutic procedures, co-morbidities, and patient care services associated with the diagnosis, as well as the titles, citations, and PubMed-abstract-weblinks to the 10 journal articles with the highest metrics.

Any flowcharts and/or block diagrams in the figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or act or carry out combinations of special purpose hardware and computer instructions.

The terminology used herein is for the purpose of describing particular embodiments only and is not intended to limit the invention. As used herein, the singular forms “a,” “an,” and “the” are intended to include the plural forms as well unless the context clearly indicates otherwise. It will further be understood that the terms “comprises” and/or “comprising,” when used in this specification, specify the presence of stated features, integers, steps, operations, elements, processes, subprocesses, and/or components but do not preclude the presence or addition of one or more other features, integers, steps, operations, elements, components, and/or groups thereof.

The corresponding structures, materials, acts, and equivalents of all means or steps plus function elements in the claims below are intended to include any structure, material, or act for performing the function in combination with other claimed elements, as specifically claimed.

The description of the present invention has been presented for purposes of illustration and description, but it is not intended to be exhaustive or limited to the invention in the form disclosed. Many modifications and variations will be apparent to those with ordinary skills in the art without departing from the scope and spirit of the invention. The embodiments are chosen and described in order to best explain the principles of the invention and the practical application, and to enable others of ordinary skills in the art to understand the invention for various embodiments with various modifications, as are suited to the particular use contemplated.

What is claimed is:

1. A system for recommending potential diagnoses, comprising:
memory; and
one or more processors, wherein the one or more processors are configured at least to;
receive user input indicating a plurality of clinical findings, the plurality of clinical findings comprising:
one or more patient demographics;
one or more patient reported symptoms;
one or more clinical examination findings;
one or more clinical laboratory results; and
one or more clinical imaging findings;
determine that one or more first clinical findings of the plurality of clinical findings are normal clinical findings;
determine that one or more second clinical findings of the plurality of clinical findings are abnormal clinical findings;
determine a plurality of diagnostic scores for respective potential diagnoses of a plurality of potential diagnoses, wherein each diagnostic score for a respective potential diagnosis of the plurality of potential diagnoses is based at least on:
one or more measurements of associations between the abnormal clinical findings and the respective potential diagnosis;
one or more measurements of inverse associations between the normal clinical findings and the respective potential diagnosis;
one or more measurements of specificity of associations between the abnormal clinical findings and the respective potential diagnosis; and
a number of patients that were diagnosed with the respective potential diagnosis during one or more periods of time; and
rank at least a portion of the plurality of potential diagnoses based at least on the plurality of diagnostic scores.

2. The system of claim 1, wherein the one or more processors are further configured to:
cause at least the portion of the plurality of potential diagnoses to be presented based at least on the ranking.

3. The system of claim 1, wherein the one or more processors are further configured to:
cause pieces of clinical guidance information associated with respective potential diagnoses of at least the portion of the plurality of potential diagnoses to be presented.

4. The system of claim 3, wherein the one or more processors are further configured to:
rank the pieces of clinical guidance information based on respective measurements of association with potential diagnoses.

5. The system of claim 3, wherein the pieces of clinical guidance information include one or more laboratory test recommendations and one or more imaging recommendations.

6. The system of claim 3, wherein the pieces of clinical guidance information include one or more diagnostic procedure recommendations, one or more recommended medications, one or more recommended therapeutic procedures, and one or more co-morbidities.

7. The system of claim 1, wherein each of the plurality of potential diagnoses corresponds to a respective standardized diagnosis code.

8. A method for recommending potential diagnoses, comprising:
receiving user input indicating a plurality of clinical findings, the plurality of clinical findings comprising:
one or more patient demographics;
one or more patient reported symptoms;
one or more clinical examination findings;
one or more clinical laboratory results; and
one or more clinical imaging findings;
determining that one or more first clinical findings of the plurality of clinical findings are normal clinical findings;
determining that one or more second clinical findings of the plurality of clinical findings are abnormal clinical findings;
determining a plurality of diagnostic scores for respective potential diagnoses of a plurality of potential diagnoses, wherein each diagnostic score for a respective potential diagnosis of the plurality of potential diagnoses is based at least on:
one or more measurements of associations between the abnormal clinical findings and the respective potential diagnosis;
one or more measurements of inverse associations between the normal clinical findings and the respective potential diagnosis;
one or more measurements of specificity of associations between the abnormal clinical findings and the respective potential diagnosis; and
a number of patients that were diagnosed with the respective potential diagnosis during one or more periods of time; and
ranking at least a portion of the plurality of potential diagnoses based at least on the plurality of diagnostic scores.

9. The method of claim 8, further comprising:
causing at least the portion of the plurality of potential diagnoses to be presented based at least on the ranking.

10. The method of claim 8, further comprising:
causing pieces of clinical guidance information associated with respective potential diagnoses of at least the portion of the plurality of potential diagnoses to be presented.

11. The method of claim 10, further comprising:
ranking the pieces of clinical guidance information based on respective measurements of association with potential diagnoses.

12. The method of claim 10, wherein the pieces of clinical guidance information include one or more laboratory test recommendations and one or more imaging recommendations.

13. The method of claim 10, wherein the pieces of clinical guidance information include one or more diagnostic procedure recommendations, one or more recommended medications, one or more recommended therapeutic procedures, and one or more co-morbidities.

14. The method of claim 8, wherein each of the plurality of potential diagnoses corresponds to a respective standardized diagnosis code.

15. A non-transitory computer-readable medium including one or more instructions that, when executed by one or more processors, cause the one or more processors to perform a method for recommending potential diagnoses, the method comprising:
receiving user input indicating a plurality of clinical findings, the plurality of clinical findings comprising:
one or more patient demographics;
one or more patient reported symptoms;
one or more clinical examination findings;
one or more clinical laboratory results; and
one or more clinical imaging findings;
determining that one or more first clinical findings of the plurality of clinical findings are normal clinical findings;
determining that one or more second clinical findings of the plurality of clinical findings are abnormal clinical findings;
determining a plurality of diagnostic scores for respective potential diagnoses of a plurality of potential diagnoses, wherein each diagnostic score for a respective potential diagnosis of the plurality of potential diagnoses is based at least on:
one or more measurements of associations between the abnormal clinical findings and the respective potential diagnosis;
one or more measurements of inverse associations between the normal clinical findings and the respective potential diagnosis;
one or more measurements of specificity of associations between the abnormal clinical findings and the respective potential diagnosis; and
a number of patients that were diagnosed with the respective potential diagnosis during one or more periods of time; and
ranking at least a portion of the plurality of potential diagnoses based at least on the plurality of diagnostic scores.

16. The non-transitory computer-readable medium of claim 15, wherein the method further comprises:
causing at least the portion of the plurality of potential diagnoses to be presented based at least on the ranking.

17. The non-transitory computer-readable medium of claim 15, wherein the method further comprises:
causing pieces of clinical guidance information associated with respective potential diagnoses of at least the portion of the plurality of potential diagnoses to be presented.

18. The non-transitory computer-readable medium of claim 17, wherein the method further comprises:
ranking the pieces of clinical guidance information based on respective measurements of association with potential diagnoses.

19. The non-transitory computer-readable medium of claim 17, wherein the pieces of clinical guidance information include one or more laboratory test recommendations and one or more imaging recommendations.

20. The non-transitory computer-readable medium of claim 17, wherein the pieces of clinical guidance information include one or more diagnostic procedure recommendations, one or more recommended medications, one or more recommended therapeutic procedures, and one or more co-morbidities.
